(19) United States
(12) Patent Application Publication
(10) Pub. No.: US 2008/0317740 A1
(43) Pub. Date: Dec. 25, 2008

(54) Use of Nk Cell Inhibition to Facilitate Persistence of Engrafted Mhc-I-Negative Cells

(76) Inventors: Bruce Blazar, Golden Valley, MN (US); Jakub Tolar, Minneapolis, MN (US); Catherine M. Verfaillie, Leuven (BE)

Correspondence Address: THOMPSON HINE L.L.P.;Intellectual Property Group, P.O. BOX 8801, DAYTON, OH, 45401-8801, US

(21) Appl. No.: 11/919,901

(22) PCT Filed: May 5, 2005
(86) PCT No.: PCT/US2005/015740

§ 371 Date: Aug. 21, 2008

(51) Int. Cl. A61K 39/395; A61K 35/12; A61K 38/00; A61P 19/00; A61N 5/00

(52) U.S. Cl. 424/130.1; 424/93.7; 424/141.1; 514/2; 607/1

(57) ABSTRACT

The present invention relates to the use of a means for inhibiting NK cell function to increase persistence and/or engraftment of MHC-I negative cells, such as MAPCs.

### REFERENCE TO RELATED APPLICATIONS

This application is a continuation-in-part of U.S. application Ser. No. 10/048,757 filed Feb. 1, 2002 which is a U.S. National Stage Application of PCT/US00/21387 filed Aug. 4, 2000 and published in English as WO 01/11011 on Feb. 15, 2001, which claims priority under 35 U.S.C. 119(e) from U.S. Provisional Application Ser. No. 60/147,324 filed Aug. 5, 1999 and 60/164,650 filed Nov. 10, 1999, which applications and publication are herein incorporated by reference.

This application is also a continuation-in-part of U.S. application Ser. No. 10/467,963 filed on Aug. 11, 2003 which is a U.S. National Stage Application of PCT/US02/04652 filed Feb. 14, 2002 and published in English as WO 02/064748 on Aug. 22, 2002, which claims priority under 35 U.S.C. 119(e) from U.S. Provisional Application Ser. Nos. 60/268,786 filed Feb. 14, 2001; 60/269,062 filed Feb. 15, 2001; 60/310,625 filed Aug. 7, 2001; and 60/343,836 filed Oct. 25, 2001, which applications and publication are herein incorporated by reference.

### STATEMENT OF GOVERNMENT RIGHTS

This work was funded by United States Grant Nos. R01-HL49997 and R01-DK58295 from the National Institutes of Health. The government may have certain rights to this invention.

### FIELD OF THE INVENTION

The present invention relates to the use of a means for inhibiting NK cell function to increase persistence and/or engraftment of MHC-I negative cells, such as multipotent adult progenitor cells (MAPCs).

### BACKGROUND OF THE INVENTION

### A. Cellular Therapy

Over the past few decades, the medical community has made great strides in the search for effective treatments and cures for some of society's most debilitating diseases. One field that has seen a great deal of advancement over the past decade is cellular therapy. Through the use of stem cells, bone marrow transplants, and therapeutic cloning, researchers and clinicians have explored ways to ultimately replace diseased or dysfunctional cells with healthy, functioning ones. However, cellular therapy still remains limited by complications such as immune rejection and graft versus host disease (GVHD). The success of cellular therapies generally depends on immune tolerance of the host to the introduced cells and their proliferation and differentiation potential. Thus, there is a need for methods to overcome the limitations of cellular therapies caused by the immunoreactivity of the host.

### B. Stem Cells

Embryonal stem (ES) cells have unlimited self-renewal and can differentiate into all tissue types. ES cells are derived from the inner cell mass of the blastocyst or primordial germ cells from a post-implantation embryo (embryonal germ cells or EG cells). ES and EG cells have been derived from mouse, and, more recently, from non-human primates and humans. When introduced into mouse blastocysts or blastocysts of other animals, ES cells can contribute to all tissues of the mouse (animal).

ES (and EG) cells can be identified by positive staining with antibodies to SSEA 1 (mouse) and SSEA 4 (human). At the molecular level, ES and EG cells express a number of transcription factors specific for these undifferentiated cells. These include Oct-4 and rex-1. Also found are the LIF-R (in mouse) and the transcription factors sox-2 and rox-1. Rox-1 and sox-2 are also expressed in non-ES cells. A hallmark of ES cells is the presence of telomerase, which provides these cells with an unlimited self-renewal potential in vitro.

Oct-4 (later designated Oct 3/4) is a transcription factor expressed in the pregastrulation embryo, early cleavage stage embryo, cells of the inner cell mass of the blastocyst, and in embryonic carcinoma (EC) cells (Nichols J., et al. 1998). Oct-4 is down-regulated when cells are induced to differentiate in vitro. Several studies have shown that Oct-4 is required for maintaining the undifferentiated phenotype of ES cells and plays a major role in determining early steps in embryogenesis and differentiation. Oct-4, in combination with Rox-1, causes transcriptional activation of the Zn-finger protein rex-1, which is also required for maintaining ES in an undifferentiated state (Rosfjord E and Rizzino A. 1997; Ben-Shushan E, et al. 1998). Likewise, sox-2, is needed together with Oct-4 to retain the undifferentiated state of ES/EC (Uwanogho D. et al. 1995) and to maintain murine (but not human) ES cells.

The Oct-4 gene (Oct 3 in humans) is transcribed into at least two splice variants in humans, Oct3A and Oct3B. The Oct3B splice variant is found in many differentiated cells whereas the Oct3A splice variant (also previously designated Oct3/4) is reported to be specific for the undifferentiated embryonic stem cell (Shimozaki et al. 2003).

Adult stem cells have been identified in most tissues. Hematopoietic stem cells are mesoderm-derived and have been purified based on cell surface markers and functional characteristics. The hematopoietic stem cell, isolated from bone marrow, blood, cord blood, fetal liver and yolk sac, is the progenitor cell that reinitiates hematopoiesis for the life of a recipient and generates multiple hematopoietic lineages. Hematopoietic stem cells can repopulate the erythroid, neutrophil-macrophage, megakaryocyte and lymphoid hemopoietic cell pool. Stem cells which differentiate only to form cells of hematopoietic lineage, however, are unable to provide a source of cells for repair of other damaged tissues, for example, heart.

Neural stem cells were initially identified in the subventricular zone and the olfactory bulb of fetal brain. Several studies in rodents, and more recently in non-human primates and humans, have shown that stem cells continue to be present in adult brain. These stem cells can proliferate in vivo and continuously regenerate at least some neuronal cells in vivo. When cultured ex vivo, neural stem cells can be induced to proliferate, as well as to differentiate into different types of neurons and glial cells. When transplanted into the brain, neural stem cells can engraft and generate neural cells and glial cells.

Mesenchymal stem cells (MSC), originally derived from the embryonal mesoderm and isolated from adult bone marrow, can differentiate to form muscle, bone, cartilage, fat, marrow stroma, and tendon. Mesoderm also differentiates into visceral mesoderm, which can give rise to cardiac muscle, smooth muscle, or blood islands consisting of endothelium and hematopoietic progenitor cells. Of the many mesenchymal stem cells that have been described, all have demonstrated limited differentiation to form only those differentiated cells generally considered to be of mesenchymal origin. To date, the best characterized mesenchymal stem cell reported is the cell isolated by Pittenger, et al. (1999) and U.S. Pat. No. 5,827,740 (SH2⁺SH4⁺ CD29⁺ CD44⁺ CD71⁺CD90⁺ CD106⁺ CD120a⁺ CD124⁺ CD14⁻ CD34⁻ CD45⁻). This cell is capable of differentiating to form a number of cell types of mesenchymal origin, but is apparently limited in differentiation potential to cells of the mesenchymal lineage.

### SUMMARY OF THE INVENTION

Natural Killer (NK) cells are characterized, in part, by cytolytic activity against cells which do not express significant major histocompatibility complex (MHC) class I molecules, such as MAPCs and embryonic stem (ES) cells. The MHC family of proteins encoded by the clustered genes of the major histocompatibility complex are expressed on cells of all higher vertebrates. They were first demonstrated in mice and called H-2 antigens (histocompatibility-2 antigens). In humans, they are sometimes also referred to as HLA antigens (human-leucocyte-associated antigens) because they were first demonstrated on leucocytes (white blood cells).

MAPC is an acronym for “multipotent adult progenitor cell” (a non ES, non EG, non germ cell) that has the capacity to differentiate into cell types of all three primitive germ layers (ectodermal, endodermal and mesodermal). Genes that have been associated with the undifferentiated state of ES cells were also found in MAPCs (e.g., telomerase, Oct 3/4, rex-1, rox-1, sox-2). Telomerase or Oct 3/4 can be recognized as genes that are primary products for the undifferentiated state. Telomerase is necessary for self-renewal.

Biologically and antigenically distinct from MSC, MAPC represents a more primitive progenitor cell population than the MSC and demonstrates differentiation capability encompassing the epithelial, endothelial, neural, myogenic, hematopoeitic, osteogenic, hepatogenic, chondrogenic and adipogenic lineages (Verfaillie, C. M. 2002; Jahagirdar, B. N., et al. 2001). MAPC thus represents a new class of non-embryonic stem cell that emulates the broad biological plasticity characteristic of ES cells, while maintaining the other characteristics that make non-embryonic cells appealing. For example, MAPCs are capable of indefinite culture without loss of differentiation potential and show efficient, long term, engraftment and differentiation along multiple developmental lineages in NOD-SCID mice without evidence of teratoma formation (Reyes, M. and C. M. Verfaillie 2001).

Embryonic stem cells and are derived from embryos and are available to the art. Specifically, embryonic stem cells are derived from embryos that develop from eggs that have been fertilized in vitro. The embryos from which human embryonic stem cells are derived are typically four or five days old and are a hollow microscopic ball of cells called the blastocyst. The blastocyst includes three structures: the trophoblast, which is the layer of cells that surrounds the blastocyst; the blastocoel, which is the hollow cavity inside the blastocyst; and the inner cell mass, which is a group of approximately 30 cells at one end of the blastocoel.

Human embryonic stem cells are isolated by transferring the inner cell mass into a culture dish that contains medium. The cells divide and spread over the surface of the dish. The inner surface of the culture dish is typically coated with a feeder layer. Recently, scientists have begun to devise ways of growing embryonic stem cells without the mouse feeder cells.

Over the course of several days, the cells of the inner cell mass proliferate and begin to crowd the culture dish. When this occurs, they are removed gently and plated into several fresh culture dishes. The process of replating the cells is repeated many times and for many months, and is called subculturing. Each cycle of subculturing the cells is referred to as a passage. After six months or more, the original 30 cells of the inner cell mass yield millions of embryonic stem cells. Embryonic stem cells that have proliferated in cell culture for six or more months without differentiating, are pluripotent, and appear genetically normal are referred to as an embryonic stem cell line. (Thomson, J. A. et al. Science 1998; 282:1145, which is incorporated herein by reference). A detailed primer on stem cells can be found at http://stemcells.nih.gov/info/basics.

One embodiment of the present invention provides a method to increase persistence of MHC-I negative cells comprising administering a population of the MHC-I negative cells and an effective amount of a means for inhibiting Natural Killer cell function to a subject, so that persistence of the MHC-I negative cells increases compared to the method without administration of the inhibiting means.

One embodiment of the present invention provides a method to increase engraftment of MHC-I negative cells comprising administering a population of the MHC-I negative cells and an effective amount of a means for inhibiting Natural Killer cell function to a subject, so that engraftment of the MHC-I negative cells increases compared to the method without administration of the inhibiting means.

Another embodiment provides a method to increase immunologic tolerance in a subject to MHC-I negative cells comprising administering a population of the MHC-I negative cells and an effective amount of a means for inhibiting Natural Killer cell function to the subject, so that immunologic tolerance to the MHC-I negative cells increases compared to the method without administration of the inhibiting means.

Yet another embodiment provides a method to inhibit rejection of MHC-I negative cells comprising administering a population of the MHC-I negative cells and an effective amount of a means for inhibiting Natural Killer cell function to a subject in need thereof, so that rejection of the MHC-I negative cells is inhibited (e.g., the cells, such as a portion of the administered population, are not rejected or survive in the subject for a longer period of time) in comparison to the method without administration of the inhibiting means.

One embodiment of the invention provides a method for treating a disease or injury in a subject comprising administering to a subject an effective amount of a population of MHC-I negative cells and an effective amount of a means for inhibiting Natural Killer cell function.

In one embodiment, the MHC-I negative cells are non-ES, non-EG, non-germ cells, wherein the non-ES, non-EG, non-germ cells and can differentiate into ectodermal, endodermal and mesodermal cell types, such as MAPCs. Such cells may express telomerase and/or Oct 3/4.

In another aspect of the invention, the MHC-I negative cells are autologous, allogeneic or xenogeneic to the subject (the recipient). In one embodiment of the invention, the non-ES, non-EG, non-germ cells are autologous, allogeneic or xenogeneic to the subject (the recipient). In another embodiment of the invention, the ES cells are allogeneic and/or xenogeneic to the subject (the recipient).

In another embodiment, the MCH-I negative cells are embryonic stem (ES) cells.

In another embodiment, the MHC-I negative cells are administered by localized injection, catheter administration, systemic injection, intraperitoneal injection, parenteral administration, oral administration, intracranial injection, intra-arterial injection, intravenous injection, intraventricular infusion, intraplacental injection, intrauterine injection, surgical intramyocardial injection, transendocardial injection, intracoronary injection, transvascular injection, intramuscular injection or via direct application to tissue surfaces during surgery or on a wound.

In one embodiment of the invention the means to inhibit Natural Killer cell function is an anti-Natural Killer cell antibody or an active fragment thereof, including a polyclonal or monoclonal antibody, or an active fragment thereof. In another embodiment the means to inhibit Natural Killer cell function is a pharmaceutical, such as a chemical compound. In one embodiment, the means to inhibit Natural Killer cell function is a protein, such as a growth factor. Another embodiment provides total body irradiation as a means to inhibit NK cell function, including a non-lethal dose of irradiation (e.g., one that does not require reconstitution of the hematopoietic system). In one embodiment the means to inhibit NK cell function is provided by the administered MHC-I negative cell which comprises an expression vector or a transgene that expresses an agent which inhibits the function of NK cells. In another embodiment a combination of means to inhibit NK cell function is administered (separately or together).

In one embodiment, the means to inhibit Natural Killer cell function is administered prior to, during and/or after administration of the non-ES, non-EG, non-germ cells. In another embodiment, the means to inhibit Natural Killer cell function is administered locally at the site of engraftment and/or systemically.

Another embodiment of the invention comprises the administration of bone marrow and/or a secondary transplant, such a heart, lung, kidney, liver transplant or a combination thereof.

In one embodiment, the subject is suffering from a disease or injury. The disease includes but is not limited to a cardiac disorder, cancer, autoimmune disease, genetic disease or hematological disease. The injury includes but is not limited to injury as a result of total body irradiation, chemoradiotherapy or physical trauma.

One embodiment of the invention provides a composition comprising a means of inhibiting NK cell function, MHC-I negative cells and a pharmaceutically acceptable carrier. Another embodiment of the invention provides a composition comprising MHC-I negative cells which provide the means of inhibiting NK cell function and a pharmaceutically acceptable carrier.

One embodiment of the invention provides for the use of a means for inhibiting Natural Killer cell (NK) function to prepare a medicament to increase persistence, increase engraftment, increase immunologic tolerance and/or decrease rejection of MHC-I negative cells. Another embodiment provides for the use of MHC-I negative cells and a means that inhibits Natural Killer cell function to prepare a medicament to increase persistence, increase engraftment, increase immunologic tolerance, decrease rejection of MHC-I negative cells and/or treat a disease and/or injury. The medicament can optionally include a physiologically acceptable carrier.

### BRIEF DESCRIPTION OF THE FIGURES

FIGS. 1A-B. MAPCs do not stimulate T cell responses in vitro. (A) BALB/c CD4⁺ T cells or (B) BALB/c CD4⁺ plus CD8⁺ T cells and irradiated, untreated C57BL/6 MAPCs or irradiated MAPCs that were pretreated with 1000 IU/ml IFN-γ for 48 hours were mixed in T cell proliferation assays. In some wells, T cells were cultured alone or with irradiated T cell-depleted C57BL/6 splenocytes. T cell proliferation was measured by ³H-thymidine uptake on day 5 and is expressed as mean±SEM.

FIG. 2. MAPCs are susceptible to NK mediated lysis in vitro. To determine whether MAPCs are susceptible targets for NK mediated killing, splenocytes from poly I:C treated C57BL/6 mice were mixed with Yac-1 cells or with MAPCs in a chromium release assay.

FIGS. 3A-E. Immune resistance to MAPC in C57BL/6, Rag2^{-/-} and Rag2^{-/-}/IL-2Rγc^{-/-} mice. (A) Using whole body imaging (WBI) of firefly luciferase bioluminescence the biodistribution of donor MAPC DL was monitored in real time on day 4 and day 30. In immunocompetent C57BL/6 mice (N=5), MAPC DL were detected on day 4, but not on day 30. (B) NK depletion did not increase MAPC DL number in the C57BL/6 mice (N=5). (C) In T- and B-cell deficient Rag2^{-/-} mice (N=5), MAPC DL were detected throughout the 30 day period, and (D) even more so in the mice which were given the anti-NK1.1 monoclonal antibody (N=5). MAPC DL were observed in the site injured by the IV injection and in the lungs, and also in intra-abdominal sites and over the long bones, as indicated by the right red circle. (E) In T, B and NK deficient Rag2^{-/-}/IL-2Rγc^{-/-} mice (N=6), MAPC DL were persistent and in 2 of 6 mice increased in number from day 4 to day 30. For example, in the third mouse from the left the luciferase signal increased 5 fold between day 4 and day 30 (red ovals), which is consistent with donor MAPC expansion. C, control.

FIG. 4. MAPCs persist in lung and differentiate into pneumocyte type I-like cells. Post-mortem, MAPCs were detected in multiple tissues including lung, liver and spleen. Shown here are donor MAPCs in the lung of the Rag2^{-/-}/IL-2Rγc^{-/-} mouse with the highest BLI 30 days after infusion. On the left, donor MAPCs appear red as a result of native DsRed2 fluorescence and nuclei are stained blue with DAPI. On the right panel, the tissue cryosection has been co-stained with anti-Aquaporin 5 antibody (to identify type 1 pneumocytes) and with DAPI. This illustrates, that donor MAPCs not only engrafted in lung, but also differentiated into alveolar type 1 pneumocytes, as indicated by arrows.

FIG. 5. TBI overcomes MAPC resistance. To determine whether MAPCs can persist under conditions of allogeneic hematopoietic stem cell transplantation, B10.BR mice were lethally irradiated and given C57BL/6 bone marrow with or without MAPC DL. 2 out of 6 representative animals (with similar BLI) are shown. Donor MAPCs were seen in the chest, abdomen, head, and extremities from day 4 through day 28 at high numbers. This suggests that total body irradiation (TBI) conditioning overcomes immune resistance and results in a widespread homing of MAPCs. C, control.

FIG. 6. Intra-arterial infusion of MAPCs results in enhanced and diverse biodistribution. To assess biodistribution of MAPCs after intravenous and intra-arterial delivery, MAPC DL (106) were infused either via tail vein (N=3) or via left cardiac ventricle into Rag2^{-/-}/IL-2Rγc^{-/-} mice (N=3). WBI of one representative animal from each group is shown at 10 weeks after either intravenous (A) or intra-arterial (B) infusion. Intra-arterial infusion lead to more diverse homing of MAPC and about 10 fold higher total body bioluminescence signals (data not shown).

### DETAILED DESCRIPTION OF THE INVENTION

MHC-I negative cells, such as MAPCs and ES cells, can be used in the methods of the invention. MAPC have the ability to regenerate all primitive germ layers (endodermal, mesodermal, and ectodermal) in vitro and in vivo. In this context they are equivalent to embryonal stem cells, and distinct from mesenchymal stem cells, which are also isolated from bone marrow. The biological potency of MAPCs has been proven in various animal models, including mouse, rat, and xenogeneic engraftment of human stem cells in rats or NOD/SCID mice (Reyes, M. and C. M. Verfaillie 2001; Jiang, Y. et al. 2002). In an elegant demonstration of the clonal potency of this cell population, single genetically marked MAPC were injected into mouse blastocysts, blastocysts implanted, and embryos developed to term (Jiang, Y. et al. 2002). Post-natal analysis in highly chimeric animals shows reconstitution of all tissues and organs, including liver. No abnormalities or organ dysfunction were observed in any of these animals.

### DEFINITIONS

As used herein, the terms below are defined by the following meanings:

“MAPC” is an acronym for “multipotent adult progenitor cell”. It refers to a non-embryonic stem cell that can differentiate to cells of all three germ layer lineages (i.e., endoderm, mesoderm and ectoderm). Like embryonic stem cells, MAPCs express Oct 3/4 (i.e., Oct-3A), rex-1, rox-1, sox-2 and telomerase. MAPC may express SSEA-4 and nanog. The term “adult,” with respect to MAPC, is non-restrictive. It refers to a non-embryonic somatic cell.

MAPCs constitutively express Oct 3/4 and high levels of telomerase (Jiang, Y. et al. 2002). MAPCs derived from human, mouse, rat or other mammals appear to be the only normal, non-malignant, somatic cell (i.e., non-germ cell) known to date to express very high levels of telomerase even in late passage cells. The telomeres are extended in MAPCs and they are karyotypically normal. Because MAPCs injected into a mammal can migrate to and assimilate within multiple organs, MAPCs are self-renewing stem cells. As such, they have utility in the repopulation of organs, either in a self-renewing state or in a differentiated state compatible with the organ of interest. They have the capacity to replace cell types that could have been damaged, died, or otherwise might have an abnormal function because of genetic or acquired disease, or, as disclosed below, may contribute to preservation of healthy cells or production of new cells in tissue.

“Multipotent,” with respect to MAPC is not limiting. It refers to the ability to give rise to cells having lineages of all three primitive germ layers (i.e., endoderm, mesoderm and ectoderm) upon differentiation.

The term “progenitor” as used in the acronym “MAPC” does not limit these cells to a particular lineage.

“Self-renewal” refers to the ability to produce replicate daughter stem cells having differentiation potential that is identical to those from which they arose. A similar term used in this context is “proliferation.”

“Expansion” refers to the propagation of a cell or cells without differentiation.

“Engraft” or “engraftment” refers to the process of cellular contact and incorporation into an existing site of interest in vivo.

Persistence refers to the ability of cells to resist rejection and remain and/or increase in number over time (e.g., days, weeks, months, years) in vivo.

The term “isolated” refers to a cell or cells which are not associated with one or more cells or one or more cellular components that are associated with the cell or cells in vivo. An “enriched population” means a relative increase in numbers of the cell of interest, such as MAPCs, relative to one or more other cell types, such as non-MAPC cell types, in vivo or in primary culture.

“Cytokines” refer to cellular factors that induce or enhance cellular movement, such as homing of MAPCs or other stem cells, progenitor cells or differentiated cells. Cytokines may also stimulate such cells to divide.

“Differentiation factors” refer to cellular factors, preferably growth factors or angiogenic factors, that induce lineage commitment.

A “subject” is a vertebrate, preferably a mammal, more preferably a human. Mammals include, but are not limited to humans, farm animals, sport animals and pets.

As used herein, “treat,” “treating” or “treatment” includes treating, preventing, ameliorating, or inhibiting an injury or disease related condition and/or a symptom of an injury or disease related condition.

An “effective amount” generally means an amount which provides the desired local or systemic effect, such as enhanced performance. For example, an effective dose is an amount sufficient to affect a beneficial or desired clinical result. Said dose could be administered in one or more administrations and could include any preselected amount of cells. The precise determination of what would be considered an effective dose may be based on factors individual to each subject, including their size, age, injury and/or disease or injury being treated and amount of time since the injury occurred or the disease began. One skilled in the art, specifically a physician, would be able to determine the number of cells that would constitute an effective dose.

“Co-administer” can include simultaneous and/or sequential administration of two or more agents.

Administered MHC-I negative cells, such as MAPCs, may contribute to generation of new tissue by differentiating into various cells in, vivo. Alternatively, or in addition, the administered cells may contribute to generation of new tissue by secreting cellular factors that aid in homing and recruitment of endogenous MAPCs or other stem cells, or other more differentiated cells. Alternatively, or in addition, the administered cells may secrete factors that act on endogenous stem or progenitor cells in the target tissue causing them to differentiate in the target site, thereby enhancing function. Further, the administered cells may secrete factors that act on stem, progenitor, or differentiated cells in the target tissue, causing them to divide. Thus, the administered cells may provide benefit via trophic influences. Examples of trophic influences include limiting inflammatory damage, limiting vascular permeability, improving cell survival at or homing of repair cells to sites of damage. Additionally, the administered cells may also provide benefit by increasing capillary density and stimulating angiogenesis. This may be achieved by production of angiogenic factors, such as VEGF, or by differentiation of the MAPCs or other stem cells and inclusion in new vessel tissue, or both. Therapeutic benefit may be achieved by a combination of the above pathways.

“Immunologic tolerance” refers to the survival (in amount and/or length of time) of foreign (e.g., allogeneic or xenogeneic) tissues, organs or cells in recipient subjects. This survival is often a result of the inhibition of a graft recipient's ability to mount an immune response that would otherwise occur in response to the introduction of foreign cells. Immune tolerance can encompass durable immunosuppression of days, weeks, months or years. Included in the definition of immunologic tolerance is NK mediated immunologic tolerance.

“Inhibit NK cell function” includes, but is not limited to, inhibiting, including reducing or eliminating, NK-cell mediated activities (e.g., NK cell mediated cell lysis and cell death), reducing or eliminating the production and/or release of cytokines by NK cells, reducing or eliminating the production and/or use of perforins, granzymes and proteoglycans by NK cells, inactivating NK cells, reducing or eliminating NK cell activation, depleting or reduce NK cells from a population of cells (e.g., cause NK cell death or inhibit the production of new NK cells such as by reducing or eliminating NK cell division), reduce or eliminate NK cell mobility (e.g., prevent them from leaving lymph nodes) and/or reduce or eliminate the ability of NK cells to recognize a target (e.g., ligand).

The terms “comprises”, “comprising”, and the like can have the meaning ascribed to them in U.S. Patent Law and can mean “includes”, “including” and the like. As used herein, “including” or “includes” or the like means including, without limitation.

### MAPCs

Human MAPCs are described in U.S. patent application Ser. Nos. 10/048,757 (PCT/US00/21387 (published as WO 01/11011)) and 10/467,963 (PCT/US02/04652 (published as WO 02/064748)), the contents of which are incorporated herein by reference for their description of MAPCs. MAPCs have been identified in other mammals. Murine MAPCs, for example, are also described in PCT/US00/21387 (published as WO 01/11011) and PCT/US02/04652 (published as WO 02/064748). Rat MAPCs are also described in WO 02/064748.

### Isolation and Growth of MAPCs

Methods of MAPC isolation for humans and mouse are known in the art. They are described in PCT/US00/21387 (published as WO 01/11011) and for rat in PCT/US02/04652 published as WO 02/064748), and these methods, along with the characterization of MAPCs disclosed therein, are incorporated herein by reference.

MAPCs were initially isolated from bone marrow, but were subsequently established from other tissues, including brain and muscle (Jiang, Y. et al., 2002). Thus, MAPCs can be isolated from multiple sources, including bone marrow, placenta, umbilical cord and cord blood, muscle, brain, liver, spinal cord, blood or skin. For example, MAPCs can be derived from bone marrow aspirates, which can be obtained by standard means available to those of skill in the art (see, for example, Muschler, G. F., et al., 1997; Batinic, D., et al., 1990). It is therefore now possible for one of skill in the art to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

MAPCs from Human Bone Marrow as Described in Pct/US00/21387

Bone marrow mononuclear cells were derived from bone marrow aspirates, which were obtained by standard means available to those of skill in the art (see, for example, Muschler, G. F., et al., 1997; Batinic, D., et al., 1990). Multipotent adult stem cells are present within the bone marrow (or other organs such as liver or brain), but do not express the common leukocyte antigen CD45 or erythroblast specific glycophorin-A (Gly-A). The mixed population of cells was subjected to a Ficoll Hypaque separation. The cells were then subjected to negative selection using anti-CD45 and anti-Gly-A antibodies, depleting the population of CD45⁺ and Gly-A⁺ cells, and the remaining approximately 0.1% of marrow mononuclear cells were then recovered. Cells could also be plated in fibronectin-coated wells and cultured as described below for 2-4 weeks to deplete the cells of CD45⁺ and Gly-A⁺ cells.

Alternatively, positive selection could be used to isolate cells via a combination of cell-specific markers. Both positive and negative selection techniques are available to those of skill in the art, and numerous monoclonal and polyclonal antibodies suitable for negative selection purposes are also available in the art (see, for example, Leukocyte Typing V, Schlossman, et al., Eds. (1995) Oxford University Press) and are commercially available from a number of sources.

Techniques for mammalian cell separation from a mixture of cell populations have also been described by Schwartz, et al., in U.S. Pat. No. 5,759,793 (magnetic separation), Basch et al., 1983 (immunoaffinity chromatography), and Wysocki and Sato, 1978 (fluorescence-activated cell sorting).

Recovered CD45⁻/GlyA⁻ cells were plated onto culture dishes coated with 5-115 ng/ml (about 7-10 ng/ml can be used) serum fibronectin or other appropriate matrix coating. Cells were maintained in Dulbecco's Minimal Essential Medium (DMEM) or other appropriate cell culture medium, supplemented with 1-50 ng/ml (about 5-15 ng/ml can be used) platelet-derived growth factor-BB (PDGF-BB), 1-50 ng/ml (about 5-15 ng/ml can be used) epidermal growth factor (EGF), 1-50 ng/ml (about 5-15 ng/ml can be used) insulin-like growth factor (IGF), or 100-10,000 IU (about 1,000 IU can be used) LIF, with 10⁻¹⁰ to 10⁻⁸ M dexamethasone (or other appropriate steroid), 2-10 μg/ml linoleic acid, and 0.05-0.15 μM ascorbic acid. Other appropriate media include, for example, MCDB, MEM, IMDM, and RPMI. Cells can either be maintained without serum, in the presence of 1-2% fetal calf serum, or, for example, in 1-2% human AB serum or autologous serum.

When re-seeded at 2×10³ cells/cm² every 3 days, >40 cell doublings were routinely obtained, and some populations underwent >70 cell doublings. Cell doubling time was 36-48 h for the initial 20-30 cell doublings. Afterwards cell-doubling time was extended to as much as 60-72 h.

Telomere length of MAPCs from 5 donors (age about 2 years to about 55 years) cultured at re-seeding densities of 2×10³ cells/cm² for 23-26 cell doublings was between 11-13 KB. This was 3-5 KB longer than telomere length of blood lymphocytes obtained from the same donors. Telomere length of cells from 2 donors evaluated after 23 and 25 cell doublings, respectively, and again after 35 cells doublings, was unchanged. The karyotype of these MAPCS was normal.

### Phenotype of Human MAPCs Under Conditions Described in PCT/US00/21387

Immunophenotypic analysis by FACS of human MAPCs obtained after 22-25 cell doublings indicated that the cells do not express CD31, CD34, CD36, CD38, CD45, CD50, CD62E and -P, HLA-DR, Muc18, STRO-1, cKit, Tie/Tek; and express low levels of CD44, HLA-class I, and β2-microglobulin, but express CD10, CD13, CD49b, CD49e, CDw90, Flk1 (N>10).

Once cells underwent >40 doublings in cultures re-seeded at about 2×10³/cm², the phenotype became more homogenous and no cell expressed HLA class-I or CD44 (n=6). When cells were grown at higher confluence, they expressed high levels of Muc18, CD44, HLA class I and β2-microglobulin, which is similar to the phenotype described for MSC(N=8) (Pittenger, 1999).

Immunhistochemistry showed that human MAPCs grown at about 2×10³/cm² seeding density expressed EGF-R, TGF-R1 and -2, BMP-R1A, PDGF-R1a and -B, and that a small subpopulation (between 1 and 10%) of MAPCs stained with anti-SSEA4 antibodies (Kannagi, R, 1983).

Using Clontech cDNA arrays the expressed gene profile of human MAPCs cultured at seeding densities of about 2×10³ cells/cm² for 22 and 26 cell doublings was determined:

A. MAPCs did not express CD31, CD36, CD62E, CD62P, CD44-H, cKit, Tie, receptors for IL1, IL3, IL6, IL 11, G CSF, GM-CSF, Epo, Flt3-L, or CNTF, and low levels of HLA-class-I, CD44-E and Muc-18 mRNA.
B. MAPCs expressed mRNA for the cytokines BMP1, BMP5, VEGF, HGF, KGF, MCP1; the cytokine receptors Flk1, EGF-R, PDGF-R1α, gp130, LIF-R, activin-R1 and -R2, TGFR-2, BMP-R1A; the adhesion receptors CD49c, CD49d, CD29; and CD10.
C. MAPCs expressed mRNA for hTRT and TRF1; the POU domain transcription factor Oct-4, sox-2 (required with Oct-4 to maintain undifferentiated state of ES/EC, Uwanogho D., 1995), sox 11 (neural development), sox 9 (chondrogenesis) (Lefebvre V., 1998); homeodeomain transcription factors: Hoxa4 and -a5 (cervical and thoracic skeleton specification; organogenesis of respiratory tract) (Packer AI, 2000), Hox-a9 (myelopoiesis) (Lawrence H, 1997), Dlx4 (specification of forebrain and peripheral structures of head) (Akimenko M A, 1994), MSX1 (embryonic mesoderm, adult heart and muscle, chondro- and osteogenesis) (Foerst-Potts L. 1997), PDX1 (pancreas) (Offield M F, 1996).
D. Presence of Oct-4, LIF-R, and hTRT mRNA was confirmed by RT-PCR.
E. In addition, RT-PCR showed that Rex-1 mRNA and Rox-1 mRNA were expressed in MAPCs.

Oct-4, Rex-1 and Rox-1 were expressed in MAPCs derived from human and murine marrow and from murine liver and brain. Human MAPCs expressed LIF-R and stained positive with SSEA-4. Finally, Oct-4, LIF-R, Rex-1 and Rox-1 mRNA levels were found to increase in human MAPCs cultured beyond 30 cell doublings, which resulted in phenotypically more homogenous cells. In contrast, MAPCs cultured at high density lost expression of these markers. This was associated with senescence before 40 cell doublings and loss of differentiation to cells other than chondroblasts, osteoblasts and adipocytes. Thus, the presence of Oct-4, combined with Rex-1, Rox-1 and sox-2 correlated with the presence of the most primitive cells in MAPCs cultures.

### Culturing MAPCs as Described in PCT/US00/21387

MAPCs isolated as described herein can be cultured using methods disclosed herein and in PCT/US00/21387, which is incorporated by reference for these methods.

Briefly, for the culture of MAPCs, culture in low-serum or serum-free medium was preferred to maintain the cells in the undifferentiated state. Serum-free medium used to culture the cells, as described herein, was supplemented as described in Table 1. Human MAPCs do not require LIF.

TABLE 1

| Insulin | 10-50 | μ/ml (10 μg/ml)* |
|---|---|---|
| Transferrin | 0-10 | μg/ml (5.5 μg/ml) |
| Selenium | 2-10 | ng/ml (5 ng/ml) |
| Bovine serum albumin (BSA) | 0.1-5 | μg/ml (0.5 μg/ml) |
| Linoleic acid | 2-10 | μg/ml (4.7 μg/ml) |
| Dexamethasone | 0.005-0.15 | μM (.01 μM) |
| L-ascorbic acid 2-phosphate | 0.1 | mM |
| Low-glucose DMEM (DMEM-LG) | 40-60% (60%) | |
| MCDB-201 | 40-60% (40%) | |
| Fetal calf serum | 0-2% | |
| Platelet-derived growth | 5-15 | ng/ml (10 ng/ml) |
| Epidermal growth factor | 5-15 | ng/ml (10 ng/ml) |
| Insulin like growth factor | 5-15 | ng/ml (10 ng/ml) |
| Leukemia inhibitory factor | 10-10,000 | IU (1,000 IU) |

*Preferred concentrations are shown in parentheses.

Addition of 10 ng/mL LIF to human MAPCs did not affect short-term cell growth (same cell doubling time till 25 cell doublings, level of Oct-4 (Oct 3/4) expression). In contrast to what was seen with human cells, when fresh murine marrow mononuclear cells, depleted on day 0 of CD45⁺ cells, were plated in MAPC culture, no growth was seen. When murine marrow mononuclear cells were plated, and cultured cells 14 days later depleted of CD45⁺ cells, cells with the morphology and phenotype similar to that of human MAPCs appeared. This suggested that factors secreted by hemopoietic cells were needed to support initial growth of murine MAPCs. When cultured with PDGF-BB and EFG alone, cell doubling was slow (>6 days) and cultures could not be maintained beyond 10 cell doublings. Addition of 10 ng/mL LIF significantly enhanced cell growth.

Once established in culture, cells could be frozen and stored as frozen stocks, using DMEM with 40% FCS and 10% DMSO. Other methods for preparing frozen stocks for cultured cells are also available to those of skill in the art.

Thus, MAPCs could be maintained and expanded in culture medium that is available to the art. Such media include, but are not limited to Dulbecco's Modified Eagle's Medium® (DMEM), DMEM F12 Medium®, Eagle's Minimum Essential Medium®, F-12K Medium®, Iscove's Modified Dulbecco's Medium® and RPMI-1640 Medium®. Many media are also available as a low-glucose formulations, with or without sodium pyruvate.

Also contemplated is supplementation of cell culture medium with mammalian sera. Sera often contain cellular factors and components that are necessary for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements, and bovine embryonic fluid. It is understood that sera can be heat-inactivated at 55-65° C. if deemed necessary to inactivate components of the complement cascade.

Additional supplements can also be used advantageously to supply the cells with the necessary trace elements for optimal growth and expansion. Such supplements include insulin, transferrin, sodium selenium and combinations thereof. These components can be included in a salt solution such as, but not limited to Hanks' Balanced Salt Solution® (HBSS), Earle's Salt Solution®, antioxidant supplements, MCDB-201® supplements, phosphate buffered saline (PBS), ascorbic acid and ascorbic acid-2-phosphate, as well as additional amino acids. Many cell culture media already contain amino acids, however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Antibiotics are also typically used in cell culture to mitigate bacterial, mycoplasmal, and fungal contamination. Typically, antibiotics or anti-mycotic compounds used are mixtures of penicillin/streptomycin, but can also include, but are not limited to amphotericin (Fungizone®), ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Antibiotic and anti-mycotic additives can be of some concern, depending on the type of work being performed. One possible situation that can arise is an antibiotic-containing media wherein bacteria are still present in the culture, but the action of the antibiotic performs a bacteriostatic rather than bacteriocidal mechanism. Also, antibiotics can interfere with the metabolism of some cell types.

Hormones can also be advantageously used in cell culture and include, but are not limited to D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine.

Lipids and lipid carriers can also be used to supplement cell culture media, depending on the type of cell and the fate of the differentiated cell. Such lipids and carriers can include, but are not limited to cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

Also contemplated is the use of feeder cell layers. Feeder cells are used to support the growth of fastidious cultured cells, particularly ES cells. Feeder cells are normal cells that have been inactivated by γ-irradiation. In culture, the feeder layer serves as a basal layer for other cells and supplies cellular factors without further growth or division of their own (Lim, J. W. and Bodnar, A., 2002). Examples of feeder layer cells are typically human diploid lung cells, mouse embryonic fibroblasts, Swiss mouse embryonic fibroblasts, but can be any post-mitotic cell that is capable of supplying cellular components and factors that are advantageous in allowing optimal growth, viability, and expansion of stem cells. In many cases, feeder cell layers are not necessary to keep the ES cells in an undifferentiated, proliferative state, as leukemia inhibitory factor (LIF) has anti-differentiation properties. Therefore, supplementation with LIF could be used to maintain MAPC in some species in an undifferentiated state.

Cells in culture can be maintained either in suspension or attached to a solid support, such as extracellular matrix components and synthetic or biopolymers. Stem cells often require additional factors that encourage their attachment to a solid support, such as type I, type II, and type IV collagen, concanavalin A, chondroitin sulfate, fibronectin, “superfibronectin” and fibronectin-like polymers, gelatin, laminin, poly-D and poly-L-lysine, thrombospondin, and vitronectin.

The maintenance conditions of stem cells can also contain cellular factors that allow stem cells, such as MAPCs, to remain in an undifferentiated form. It is advantageous under conditions where the cell must remain in an undifferentiated state of self-renewal for the medium to contain epidermal growth factor (EGF), platelet derived growth factor (PDGF), leukemia inhibitory factor (LIF; in selected species), and combinations thereof. It is apparent to those skilled in the art that supplements that allow the cell to self-renew but not differentiate must be removed from the culture medium prior to differentiation.

Stem cell lines and other cells can benefit from co-culturing with another cell type. Such co-culturing methods arise from the observation that certain cells can supply yet-unidentified cellular factors that allow the stem cell to differentiate into a specific lineage or cell type. These cellular factors can also induce expression of cell-surface receptors, some of which can be readily identified by monoclonal antibodies. Generally, cells for co-culturing are selected based on the type of lineage one skilled in the art wishes to induce, and it is within the capabilities of the skilled artisan to select the appropriate cells for co-culture.

Methods of identifying and subsequently separating differentiated cells from their undifferentiated counterparts can be carried out by methods well known in the art. Cells that have been induced to differentiate can be identified by selectively culturing cells under conditions whereby differentiated cells outnumber undifferentiated cells. Similarly, differentiated cells can be identified by morphological changes and characteristics that are not present on their undifferentiated counterparts, such as cell size, the number of cellular processes (i.e. formation of dendrites and/or branches), and the complexity of intracellular organelle distribution. Also contemplated are methods of identifying differentiated cells by their expression of specific cell-surface markers such as cellular receptors and transmembrane proteins. Monoclonal antibodies against these cell-surface markers can be used to identify differentiated cells. Detection of these cells can be achieved through fluorescence activated cell sorting (FACS), and enzyme-linked immunosorbent assay (ELISA). From the standpoint of transcriptional upregulation of specific genes, differentiated cells often display levels of gene expression that are different from undifferentiated cells. Reverse-transcription polymerase chain reaction (RT-PCR) can also be used to monitor changes in gene expression in response to differentiation. In addition, whole genome analysis using microarray technology can be used to identify differentiated cells.

Accordingly, once differentiated cells are identified, they can be separated from their undifferentiated counterparts, if necessary. The methods of identification detailed above also provide methods of separation, such as FACS, preferential cell culture methods, ELISA, magnetic beads, and combinations thereof. A preferred embodiment of the invention envisions the use of FACS to identify and separate cells based on cell-surface antigen expression.

Additional Culture Methods

In additional experiments it has been found that the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 3-5%, can be used at any time during the isolation, growth and differentiation of MAPCs in culture.

### Natural Killer Cell Function

Natural Killer (NK) cells are a subset of large granular lymphocytes that are cytotoxic cells. NK cells make up approximately 15% of the human white blood cells and are characterized by cytolytic activity against cells which do not express major histocompatibility complex (MHC) class I molecules (e.g., tumor cells or virally infected cells). They kill (lyse) target cells using perforins, granzymes and proteoglycans. They are called “natural” killers because they do not need to recognize a specific antigen before lysing cells. NK cells have no immunological memory and are independent of the adaptive immune system.

NK cell activity and NK cell count are not the same. NK cells may be present in sufficient numbers, but unless they are activated they are ineffective. One function of NK cells is to reject foreign materials, such as histo-incompatible marrow, stem cell grafts (e.g., pluripotent, muscle, neural, liver, and other stem cell types) and organ transplants resulting in the failure of a recipient's body to accept transplanted cells or a tissue or organ. Activated NK cells also produce a variety of cytokines, including interferons (IFN-γ), interleukins, TNF (Tumor Necrosis Factor, e.g., TNF-α), hematopoietic cell growth factors and other growth factors.

The present invention provides means for the inhibition of NK cell-mediated function(s) to promote cell engraftment and/or persistence, including MAPC engraftment. Inhibiting NK cell function includes inhibiting, including reducing or eliminating, NK-cell mediated activities (e.g., NK cell mediated cell lysis and cell death). Inhibiting NK cell functions also includes but is not limited to reducing or eliminating the production and/or release of cytokines by NK cells, reducing or eliminating the production and/or use of perforins, granzymes and proteoglycans by NK cells, inactivating NK cells, reducing or eliminating NK cell activation, depleting NK cells from a population of cells (e.g., cause NK cell death or reduce or eliminate the production of new NK cells), reduce or eliminate NK cell division, reduce or eliminate NK cell mobility (e.g., prevent them from leaving lymph nodes) and/or reduce or eliminate the ability of NK cells to recognize a target (e.g., ligand).

There are many tests available to one of skill in the art to test for inhibition of NK cell activity, including trypan blue staining of dead cells after an in vitro cytotoxicity assay to determine abrogation of cytolysis of NK-cell-specific target cells (e.g., Yac-1 cells).

A. Means for Inhibiting NK Cell Function

In one embodiment of the invention at least one means for inhibiting NK cell function, including inhibition of NK cell-mediated cytotoxicity, is administered. NK cell function can be negated by NK depletion using either genetic (recipients deficient in NK cells) or epigenetic (in vivo depletion/inactivation with, for example, an anti-NK antibody) means. Any material capable of inhibiting NK cell function can be used (e.g., multimeric compounds that bind to P-Selectin Glycoprotein 1 (PSGL-1) on the surface of T cells or NK cells (U.S. Pat. Pub. No. 2004/0116333) or modulation of SH2-containing inositol phophatase (SHIP) expression or function (U.S. Pat. Pub. No. 2002/0165192)). Any means/agent including but not limited to, chemical (e.g., a chemical compound, including but not limited to a pharmaceutical, drug, small molecule), protein (e.g., anti-NK cell antibody), peptide, microorganism, biologic, nucleic acid (including genes coding for recombinant proteins, or antibodies), or genetic construct (e.g., vectors, such as expression vectors, including but not limited to expression vectors which lead to expression of an antagonist against NK cell activity) can be used to inhibit NK cell function.

Additionally, a means, such as an agent which can cross-link LAIR-1 molecules on NK cells may be used to inhibit NK cell function. Also, irradiation (lethal, sub-lethal, and/or localized irradiation) may be used to inhibit NK cell function. In one embodiment, the means for inhibiting NK cell function is an antibody which is reactive with Natural Killer cells. Additionally, a means for inhibiting NK cell function can include agents that modulate the immune system, such as those developed for immunosuppression (see below for further discussion). It should be noted that any of these means/agents can be used alone or in combination.

1. Anti-NK Cell Antibodies

There are several antibodies available in the art which inhibit NK cell function, including but not limited to anti-human thymocyte globulin (ATG; U.S. Pat. No. 6,296,846), TM-β1 (anti-IL-2 receptor β chain Ab), anti-asialo-GM1 (immunogen is the glycolipid GA1), anti-NK1.1 antibodies or monoclonal anti-NK-cell antibodies (5E6; Pharmingen, Piscataway, N.J.). Additionally, antibodies directed against, for example, a natural cytotoxicity receptor (NCR), including, for example, NKp46, or an antibodies directed against a leukocyte-associated Ig like receptor family, including, for example, LAIR-1, or antibodies directed against a member of the killer cell immunoglobulin-like receptor (KIR) family, including, for example, KIR2DL1, KIR2DL2 or KR2DL3 are available to the art worker or can be made by methods available to an art worker and are useful in the present invention.

Also within the scope of the invention is the production and use of polyclonal or monoclonal antibodies, or active fragments thereof which recognize antigens expressed by NK cells including but not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above, which recognize NK cell antigens, such as cell surface markers. Additionally, the antibody may be coupled to a toxin. Antibodies directed to antigens of NK cells may be used to specifically inhibit NK cell function. Such antibodies may be used in conjunction with MAPC administration, with irradiation, including sub-lethal irradiation, and/or cytotoxic drugs and/or immunosuppressive drugs.

All antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., 1985; Novotny and Haber, 1985).

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody that includes the variable domain complementarity determining regions (CDR), and the like forms, all of which fall under the broad term “antibody,” as used herein. The present invention contemplates the use of any specificity of an antibody, polyclonal or monoclonal, and is not limited to antibodies that recognize and immunoreact with a specific epitope.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments, which are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments retain some ability to selectively bind with its antigen or receptor and are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule that can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

(5) Single chain antibody (“SCA”), defined as a genetically engineered molecule containing the variable region of the light chain and the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., 1993).

The preparation of polyclonal antibodies is well-known to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference. For example, for the production of polyclonal antibodies, various host animals can be immunized by injection with purified or partially purified NK cells or proteins associated therewith. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

The preparation of monoclonal antibodies likewise is conventional. See, for example, Kohler & Milstein, 1975; Coligan, et al., sections 2.5.1-2.6.7; and Harlow, et al., in: Antibodies: A Laboratory Manual, page 726 (Cold Spring Harbor Pub. (1988)), which are hereby incorporated by reference. Methods of in vitro and in vivo manipulation of monoclonal antibodies are also available to those skilled in the art. For example, the monoclonal antibodies to be used in accordance with the present invention may be made by the hybridoma method first described by Kohler and Milstein (1975), or they may be made by recombinant methods, for example, as described in U.S. Pat. No. 4,816,567. The monoclonal antibodies for use with the present invention may also be isolated from antibody libraries using the techniques described in Clackson et al. (1991), as well as in Marks et al. (1991).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Another method for generating antibodies involves a Selected Lymphocyte Antibody Method (SLAM). The SLAM technology permits the generation, isolation and manipulation of monoclonal antibodies without the process of hybridoma generation. The methodology principally involves the growth of antibody forming cells, the physical selection of specifically selected antibody forming cells, the isolation of the genes encoding the antibody and the subsequent cloning and expression of those genes.

More specifically, an animal is immunized with a source of specific antigen. The animal can be a rabbit, mouse, rat, or any other convenient animal. This immunization may consist of purified protein, in either native or recombinant form, peptides, DNA encoding the protein of interest or cells expressing the protein of interest. After a suitable period, during which antibodies can be detected in the serum of the animal (usually weeks to months), blood, spleen or other tissues are harvested from the animal. Lymphocytes are isolated from the blood and cultured under specific conditions to generate antibody-forming cells, with antibody being secreted into the culture medium. These cells are detected by any of several means (complement mediated lysis of antigen-bearing cells, fluorescence detection or other) and then isolated using micromanipulation technology. The individual antibody forming cells are then processed for eventual single cell PCR to obtain the expressed Heavy and Light chain genes that encode the specific antibody. Once obtained and sequenced, these genes are cloned into an appropriate expression vector and recombinant, monoclonal antibody produced in a heterologous cell system. These antibodies are then purified via standard methodologies such as the use of protein A affinity columns. These types of methods are further described in Babcook, et al. 1996; U.S. Pat. No. 5,627,052; and PCT WO 92/02551 by Schrader.

Another method involves humanizing a monoclonal antibody by recombinant means to generate antibodies containing human specific and recognizable sequences. See, for review, Holmes, et al. (1997) and Vaswani, et al. (1998).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81, 6851-6855 (1984).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab monovalent fragments. Alternatively, an enzymatic cleavage using pepsin produces two monovalent Fab′ fragments and an Fc fragment directly. These methods are described, for example, in U.S. Pat. No. 4,036,945 and No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird et al. (1988); Ladner, et al, U.S. Pat. No. 4,946,778; and Pack, et al. (1993).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention further contemplates human and humanized forms of non-human (e.g. murine) antibodies. Such humanized antibodies can be chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies can comprise substantially all of at least one, and typically two, variable domains, in which all or substantially all of the CDR regions correspond to those of a non-human immunoglobulin and all or substantially all of the Fv regions are those of a human immunoglobulin consensus sequence. The humanized antibody optimally also will comprise at least a portion of an immunoglobulin constant region (Fc), typically that of a human immunoglobulin. For further details, see: Jones et al. (1986); Reichmann et al. (1988); Presta (1992); Holmes (1997) and Vaswani, et al. (1998); U.S. Pat. Nos. 4,816,567 and 6,331,415; PCT/GB84/00094; PCT/US86/02269; PCT/US89/00077; PCT/US88/02514; and WO91/09967, each of which is incorporated herein by reference in its entirety.

The invention also provides methods of mutating antibodies to optimize their affinity, selectivity, binding strength or other desirable property. A mutant antibody refers to an amino acid sequence variant of an antibody. In general, one or more of the amino acid residues in the mutant antibody is different from what is present in the reference antibody. Such mutant antibodies necessarily have less than 100% sequence identity or similarity with the reference amino acid sequence. In general, mutant antibodies have at least 75% amino acid sequence identity or similarity with the amino acid sequence of either the heavy or light chain variable domain of the reference antibody. Preferably, mutant antibodies have at least 80%, more preferably at least 85%, even more preferably at least 90%, and most preferably at least 95% amino acid sequence identity or similarity with the amino acid sequence of either the heavy or light chain variable domain of the reference antibody.

The antibodies of the invention are isolated antibodies. An isolated antibody is one that has been identified and separated and/or recovered from the environment in which it was produced. In general, the isolated antibodies of the invention are substantially free of at least some contaminant components of the environment in which they were produced. Contaminant components of its production environment are materials that would interfere with diagnostic or therapeutic uses for the antibody, and may include cells, enzymes, hormones, and other proteinaceous or nonproteinaceous solutes. The term “isolated antibody” also includes antibodies within recombinant cells because at least one component of the antibody's natural environment will not be present. Ordinarily, however, isolated antibody will be prepared by at least one purification step.

If desired, the antibodies of the invention can be purified by any available procedure. For example, the antibodies can be affinity purified by binding an antibody preparation to a solid support to which the antigen used to raise the antibodies is bound. After washing off contaminants, the antibody can be eluted by known procedures. Those of skill in the art will know of various techniques common in the immunology arts for purification and/or concentration of polyclonal antibodies, as well as monoclonal antibodies (see for example, Coligan, et al., Unit 9, Current Protocols in Immunology, Wiley Interscience, 1991, incorporated by reference).

In some embodiments, the antibody will be purified as measurable by at least three different methods: 1) to greater than 95% by weight of antibody as determined by the Lowry method, and most preferably more than 99% by weight; 2) to a degree sufficient to obtain at least 15 residues of N-terminal or internal amino acid sequence by use of a spinning cup sequenator; or 3) to homogeneity by SDS-PAGE under reducing or non-reducing conditions using Coomassie blue or, preferably, silver stain.

Additionally, the anti-NK antibodies or active fragments thereof can be modified by the attachment of a toxic agent so that the resulting molecule can be used to kill or inactivate cells which express the corresponding antigen. Any method available to the art can be used to couple antibodies to a toxic agent, including the generation of fusion proteins by recombinant DNA technology.

2. Immunosuppressive Pathways

A. Pathology and Immunology of Graft Rejection

Organ transplantation is accompanied by a complex series of immunologic responses. These are generally categorized as inflammation, immunity, and tissue repair and structural reinforcement of damaged tissues. Inflammation in the transplantation site is mediated by macrophages, T cells and proinflammatory mediators (e.g., IL-2). This is followed by activation of biochemical cascades (e.g., classic complement cascade) resulting in elaboration of bioactive intermediates such as C3a and C5a. After donor cells have been recognized and rejected by the immune system, macrophages, endothelial cells, smooth muscle cells, and fibroblasts begin to promote repair and structural reinforcement of damaged cells.

Rejection results when a pathologic and inflammatory response develops or when repair and remodeling of tissues fails. In hyperacute rejection, transplant patients are serologically presensitized to alloantigens (i.e., graft antigens are recognized as foreign). Hyperacute rejection may develop within minutes to hours of graft implantation.

In acute rejection, graft alloantigens are encountered by T cells, with resulting cytokine (and possibly antibody) release that then leads to tissue distortion, vascular insufficiency, and cell destruction. These processes can occur within 24 hours after graft implantation and continue over a period of days to weeks.

In chronic rejection, pathologic tissue remodeling and reinforcement occurs. Blood flow is reduced, which leads to regional tissue ischemia, fibrosis, and cell death.

The control of acute rejection has been the primary aim of immunosuppression, thereby allowing tissue repair to progress. The use of combination immunosuppressive therapy has evolved over a number of years.

B. Examples of Immunosuppressive Drugs for Use in the Invention

Azathioprine—is a derivative of 6-mercaptopurine. It functions as an antimetabolite to decrease DNA and RNA synthesis and is used for maintenance immunosuppression.

Corticosteroids prevent interleukin (IL)-1 and IL-6 production by macrophages and inhibit all stages of T-cell activation. This agent is used for induction, maintenance immunosuppression, and acute rejection.

Cyclosporine is a polypeptide of 11 amino acids of fungal origin and is active against helper T cells, preventing the production of IL-2 via calcineurin inhibition (binds to cyclophilin protein). This agent is used for induction and maintenance immunosuppression.

Tacrolimus is a macrolide antibiotic and is active against helper T cells, preventing the production of IL-2 via calcineurin inhibition (binds to tacrolimus-binding protein instead of cyclophilin protein). This agent is used for maintenance immunosuppression and for rescue therapy in patients with refractory rejection under cyclosporine-based therapy.

Mycophenolate mofetil inhibits the enzyme inosine monophosphate dehydrogenase (required for guanosine synthesis) and impairs B- and T-cell proliferation, sparing other rapidly dividing cells (because of the presence of guanosine salvage pathways in other cells). This agent is used for maintenance immunosuppression and chronic rejection.

Sirolimus is a macrolide antibiotic that binds the FK-binding protein, but its mechanism of action is via the “target of Rapamune,” or TOR. It inhibits G1- to S-phase cell division and, therefore, cell proliferation. This agent is used for maintenance immunosuppression and chronic rejection.

C. Examples of Biologicals Mediating Immunosuppression for Use in the Invention

Polyclonal antibodies (e.g., anti-thymocyte globulins): These agents are derived by injecting animals with human lymphoid cells, then harvesting and purifying the resultant antibody. Polyclonal antibodies induce the complement lysis of lymphocytes and uptake of lymphocytes by the reticuloendothelial system and mask the lymphoid cell-surface receptors. Preparations include horse anti-thymocyte globulin (Atgam) and rabbit anti-thymocyte globulin (Thymoglobulin).

Muromonab-CD3: is a murine monoclonal antibody of immunoglobulin 2A clones to the CD3 portion of the T-cell receptor. It blocks T-cell function and has limited reactions with other tissues or cells. This agent is used for induction and acute rejection (primary treatment or steroid-resistant).

Basiliximab (Simulect) and daclizumab (Zenapax): are humanized monoclonal antibodies that target the IL-2 receptor. Clinically, both agents are very similar, and both are used for induction of immunosuppression.

### Therapeutic Uses of MHC-I Negative Cells and Means for Inhibiting NK Cell Activity

Means for inhibiting NK cell activity are useful for promoting stem cell, e.g., MAPC, engraftment, persistence and/or donor-specific tolerance for the enhancement of transplantation success or outcomes. The promotion of stem cell engraftment, persistent and/or tolerance is an issue not only in cell transplantation, i.e., to promote acceptance of the cells by the transplant recipient, but also in the treatment of a variety of diseases and injuries.

MHC-I negative cells, such as MAPCs and ES cells, and a means for inhibiting NK cell activity can be used for preclinical, such as in large animal models of disease or injury, and clinical, such as therapeutic, settings (Use of MAPCs isolated from humans and mice are described in PCT/US0021387 (published as WO 01/11011) and from rat in PCT/US02/04652 (published as WO 02/064748), and these uses are incorporated herein by reference.)

For example, MAPCs and ES cells can differentiate to form all three germ cell layers. For example, MAPCs can be induced to differentiate into chondrocytes, hepatocytes, endothelial cells, cardiomyocytes, smooth muscle cells, and neural cells. As such, MAPCs, ES cells or progeny derived therefrom can be used to treat essentially any injury or disease, particularly a disease associated with pathological change in an organ or tissue physiology or morphology which is amenable to treatment by cell, tissue or organ transplantation in any mammalian species, preferably in a human. Administered MAPCs or ES cells may contribute to the generation of new tissue by differentiating in vivo. For example, MAPCs can be used to repopulate depleted or damaged heart muscle cells, or cells of any other organ or tissue, by either direct injection into the area of tissue damage or by systemic injection, followed by allowing the cells to home to the tissue or organ. This method can be particularly effective if combined with angiogenesis. Both methods of injection and methods for promoting angiogenesis are known to those of skill in the art.

Diseases treatable by MHC-I negative cell based therapy include but are not limited to renal, pancreatic, cardiac, hepatic, hematological, genetic, pulmonary, brain, gastrointestinal, muscular, lung, endocrine, neural, metabolic, dermal, cosmetic, opthalmological, and vascular diseases.

Examples of renal diseases which can be treated using MHC-I negative cells or progeny derived therefrom, include but are not limited to acute kidney failure, acute nephritic syndrome, analgesic nephropathy, atheroembolic kidney disease, chronic kidney failure, chronic nephritis, congenital nephrotic syndrome, end-stage kidney disease, Goodpasture's syndrome, IgM mesangial proliferative glomerulonephritis, interstitial nephritis, kidney cancer, kidney damage, kidney infection, kidney injury, kidney stones, lupus nephritis, membranoproliferative glomerulonephritis I, membranoproliferative glomerulonephritis II, membranous nephropathy, necrotizing glomerulonephritis, nephroblastoma, nephrocalcinosis, nephrogenic diabetes insipidus, IgA-mediated nephropathy, nephrosis, nephrotic syndrome, polycystic kidney disease, post-streptococcal glomerulonephritis, reflux nephropathy, renal artery embolism, renal artery stenosis, renal papillary necrosis, renal tubular acidosis type I, renal tubular acidosis type II, renal underperfusion and renal vein thrombosis.

Examples of lung diseases which can be treated using MHC-I negative cells or progeny derived therefrom include but are not limited to environmental lung disease, occupational lung disease (e.g., mesothioloma), asthma, BOOP, chronic bronchitis, COPD (chronic obstructive pulmonary disease), emphysema, interstitial lung disease, pulmonary fibrosis, sarcoidosis, asbestosis, aspergilloma, aspergillosis, aspergillosis-acute invasive, atelectasis, eosinophilic pneumonia, lung cancer, metastatic lung cancer, necrotizing pneumonia, pleural effusion, pneumoconiosis, pneumocystosis, pneumonia, pneumonia in immunodeficient patient, pneumothorax, pulmonary actinomycosis, pulmonary alveolar proteinosis, pulmonary anthrax, pulmonary arteriovenous malformation, pulmonary edema, pulmonary embolus, pulmonary histiocytosis X (eosinophilic granuloma), pulmonary hypertension, pulmonary nocardiosis, pulmonary tuberculosis, pulmonary veno-occlusive disease, and rheumatoid lung disease. These diseases can all cause damage/injury to lung tissue.

Examples of pancreatic diseases which can be treated using MHC-I negative cells or progeny derived therefrom include but are not limited to Type I or Type II diabetes.

Examples of hepatic diseases which can be treated using MHC-I negative cells or progeny derived therefrom include but are not limited to hepatitis C infection, hepatic cirrhosis, primary sclerosing cholangitis, NASH, hepatocellular carcinoma, alcoholic liver disease, and hepatitis B.

In the case of cardiac disease, diseases which can be treated using MHC-I negative cells or progeny therefrom include but are not limited to myocarditis, cardiomyopathy, heart failure, damage caused by heart attacks, hypertension, atherosclerosis or heart valve dysfunction. Progeny can include cardiomyocytes that repopulate the injured tissue or endothelial cells that provide neo-vascularization to the tissue.

MHC-I negative cells can also be administered to provide vasculature in subjects suffering from a loss and/or function of vascularization as a result of physical or disease related damage. Disease states characterized by a loss of vascularization and/or function, and that benefit from methods of the present invention include vascular conditions, such as ischemia (including ischemia-reperfusion injury), congestive heart failure, peripheral vasculature disorder, coronary vascular disease, diabetic ulcers, pressure ulcers, hypertension, stroke, aneurysm, thrombosis, arrhythmia, tachycardia, or surgical or physical (e.g., wounding) trauma.

In one embodiment, MHC-I negative cell-based therapies can be used to treat damage resulting from disease states including but not limited to congestive heart failure, coronary artery disease, myocardial infarction, myocardial ischemia, effects of atherosclerosis or hypertension, cardiomyopathy, cardiac arrhythmias, infective myocarditis, hypersensitivity myocarditis, autoimmune endocarditis, and congenital heart disease.

For example, cardiac injury, such as MI, promotes tissue responses that enhance myogenesis using implanted MAPCs. Thus, administration of MAPCs can, for example, reduce the degree of scar formation, augment ventricular function, and compensate for weakened cardiac muscle, thereby improving cardiac function. New muscle is thereby created within an infarcted myocardial segment. Preferably, MHC-I negative cells, such as MAPCs or ES cells, can directly infiltrate into the zone of infarcted tissue. In preferred embodiments, engraftment of MHC-I negative cells is within cardiac muscle in acute myocardial infarction.

In the case of degenerative myocardial disease, MHC-I negative cells can provide for both myocyte replacement and stimulation of angiogenesis. Improved cardiac function can be indicated, for example, by increased perfusion. This therapy can be used as a stand alone therapy or in conjunction with revascularization therapies. MHC-I negative cells, such as MAPCs or ES cells, also offer the advantage of forming vascular structures to furnish and supply blood to the emerging cardiac muscle mass.

MHC-I negative cell-based therapies are not limited to improvement of cardiac muscle pathologies, but can be extended to any type of muscular disorder in which the primary pathology is loss of striated muscle mass and/or function. This would include but is not limited to muscle degeneration, mitochondrial diseases, myoclonus, seizure disorders, tremors, muscular dystrophies, trauma, myasthenia gravis, and toxin-induced muscle abnormalities. Thus, in another embodiment, the present invention comprises methods of increasing striated muscle tissue mass by contacting a suitable amount of MHC-I negative cells with existing striated muscle tissue and generating viable striated muscle tissue.

Examples of hematological and/or genetic diseases which can be treated using MHC-I negative cells or progeny derived therefrom include but are not limited to coagulation disorders/coagulation factor deficiencies such as hemophilia, thallassemia, chronic granulomatous disease and lysosomal storage diseases/enzyme deficiencies such as Gaucher disease.

In one embodiment, hematopoietic diseases include, but are not limited to:
leukemias (leukemia is a cancer of the blood immune system, whose cells are called leukocytes or white cells) including but not limited to Acute Leukemia, Acute Lymphoblastic Leukemia (ALL), Acute Myelogenous Leukemia (AML), Acute Biphenotypic Leukemia, Acute Undifferentiated Leukemia, Chronic Leukemia, Chronic Myelogenous Leukemia (CML), Chronic Lymphocytic Leukemia (CLL), Juvenile Chronic Myelogenous Leukemia (JCML), Juvenile Myelomonocytic Leukemia (JMML);
myelodysplastic Syndromes (myelodysplasia is sometimes called pre-leukemia) including but not limited to Refractory Anemia (RA), Refractory Anemia with Ringed Sideroblasts (RARS), Refractory Anemia with Excess Blasts (RAEB), Refractory Anemia with Excess Blasts in Transformation (RAEB-T), Chronic Myelomonocytic Leukemia (CMML);
lymphomas (lymphoma is a cancer of the leukocytes that circulate in the blood and lymph vessels) including but not limited to Hodgkin's Lymphoma, Non-Hodgkin's Lymphoma, Burkitt's Lymphoma;
inherited red cell (Erythrocyte) abnormalities (red cells contain hemoglobin and carry oxygen to the body) including but not limited to Beta Thalassemia Major (also known as Cooley's Anemia), Blackfan-Diamond Anemia, Pure Red Cell Aplasia, Sickle Cell Disease;
other disorders of blood cell proliferation including but not limited to anemias (anemias are deficiencies or malformations of red cells) including but not limited to severe Aplastic Anemia, Congenital Dyserythropoietic Anemia, and Fanconi Anemia, Paroxysmal Nocturnal Hemoglobinuria (PNH),
inherited platelet abnormalities (platelets are small blood cells needed for clotting) including but not limited to Amegakaryocytosis/Congenital Thrombocytopenia, Glanzmann Thrombasthenia, Myeloproliferative Disorders, Acute Myelofibrosis, Agnogenic Myeloid Metaplasia (Myelofibrosis), Polycythemia Vera, Essential Thrombocythemia;
inherited immune system disorders—Severe Combined Immunodeficiency (SCID) including but not limited to SCID with Adenosine Deaminase Deficiency (ADA-SCID), SCID which is X-linked, SCID with absence of T & B Cells, SCID with absence of T Cells, Normal B Cells, Omenn Syndrome;
inherited immune system disorders—Neutropenias including but not limited to Kostmann Syndrome, Myelokathexis;
other inherited immune system disorders including but not limited to Ataxia-Telangiectasia, Bare Lymphocyte Syndrome,

### Common Variable Immunodeficiency, DiGeorge Syndrome, Leukocyte Adhesion Deficiency;

lymphoproliferative disorders (LPD) including but not limited to Lymphoproliferative Disorder, X-linked (also known as Epstein-Barr Virus Susceptibility), Wiskott-Aldrich Syndrome;
phagocyte Disorders (phagocytes are immune system cells that can engulf and kill foreign organisms) including but not limited to Chediak-Higashi Syndrome, Chronic Granulomatous Disease, Neutrophil Actin Deficiency, Reticular Dysgenesis;
cancers in the bone marrow (plasma cell disorders) including but not limited Multiple Myeloma, Plasma Cell Leukemia, Waldenstrom's Macroglobulinemia; and
other cancers (not originating in the blood system) including but not limited to Neuroblastoma.

Examples of neurological disorders which can be treated using MAPCs or progeny derived therefrom include but are not limited to Parkinson's, ALS, and Huntington's disease.

Examples of other diseases or disease conditions in which the methods of the present invention are useful include but are not limited to cancer, including lymphoma (e.g., non-Hodgkin's lymphoma), acute and chronic leukemias (e.g., chronic myelogenous leukemia) or other hematological diseases/disorders (e.g., aplastic anemia, sickle cell anemia, thalassemia), solid organ, tissue or cellular transplantation, immunodeficiency, diabetes, multiple sclerosis, sickle cell anemia and other autoimmune disease states, Graft Versus Host Disease (GVHD) or a genetic deficiency or impairment (e.g., Hurler's syndrome, Fanconi Anemia (FA))

Injuries that can be treated using MHC-I negative cells include but are not limited to injury as a result of disease, physical (wound) or surgical trauma and tissues injured by chemotherapy or irradiation used for conditioning for hematopoietic stem cell transplantation.

### Administration of MHC-I Negative Cells and Means for Inhibiting NK Cell Function

A. Administration of MHC-I Negative Cells

MHC-I negative cells, such as MAPCs or ES cells, or their differentiated progeny, can be administered to a subject by a variety of methods available to the art, including but not limited to localized injection, catheter administration, systemic injection, intraperitoneal injection, parenteral administration, oral administration, intracranial injection, intra-arterial injection (as discussed in the Examples section below, intra-arterial injection provides for more diverse homing/greater bio-distribution than intravenous injection), intravenous injection, intraventricular infusion, intraplacental injection, intrauterine injection, surgical intramyocardial injection, transendocardial injection, transvascular injection, intracoronary injection, transvascular injection, intramuscular injection, surgical injection into a tissue of interest or via direct application to tissue surfaces (e.g., during surgery or on a wound).

Intravenous injection is the simplest method of cell administration; however a greater degree of dependence on homing of the stem cells is required for them to reach the tissue of interest (e.g., lung). Carefully controlled dosing, which is readily determined by one skilled in the art, enhances this method of administration.

MHC-I negative cells can be administered either peripherally or locally through the circulatory system. “Homing” of stem cells to the injured tissues would concentrate the implanted cells in an environment favorable to their growth and function. Pre-treatment of a patient with cytokine(s) to promote homing is another alternative contemplated in the methods of the present invention. Where homing signals may be less intense, injection of the cells directly into the lung may produce a more favorable outcome. Certain cytokines (e.g., cellular factors that induce or enhance cellular movement, such as homing of MHC-I negative cells, such as MAPCs or other stem cells, progenitor cells or differentiated cells) can enhance the migration of MHC-I negative cells or their differentiated counterparts to the site of damaged lung tissue. Cytokines include, but are not limited to, stromal cell derived factor-1 (SDF-1), stem cell factor (SCF) and granulocyte-colony stimulating factor (G-CSF). Cytokines also include any which promote the expression of endothelial adhesion molecules, such as ICAMs, VCAMs, and others, which facilitate the homing process.

Differentiation of MHC-I negative cells to a phenotype characteristic of a desired tissue can be enhanced when differentiation factors are employed, e.g., factors promoting formation of the desired lung tissue.

Viability of newly forming tissues can be enhanced by angiogenesis. Factors promoting angiogenesis include but are not limited to VEGF, aFGF, angiogenin, angiotensin-1 and -2, betacellulin, bFGF, Factor X and Xa, HB-EGF, PDGF, angiomodulin, angiotropin, angiopoetin-1, prostaglandin E1 and E2, steroids, heparin, 1-butyryl-glycerol, nicotinic amide.

Factors that decrease apoptosis can also promote the formation of new tissue, such as lung epithelium. Factors that decrease apoptosis include but are not limited to β-blockers, angiotensin-converting enzyme inhibitors (ACE inhibitors), AKT, HIF, carvedilol, angiotensin II type 1 receptor antagonists, caspase inhibitors, cariporide, and eniporide.

Exogenous factors (e.g., cytokines, differentiation factors (e.g., cellular factors, preferably growth factors or angiogenic factors that induce lineage commitment), angiogenesis factors and anti-apoptosis factors) can be administered prior to, after or concomitantly with MHC-I negative cells or their differentiated progeny (e.g., alveolar type II epithelial or epithelial like cells). For example, a form of concomitant administration would comprise combining a factor of interest in the MAPC or ES cell suspension media prior to administration. Doses for administration(s) are variable and may include an initial administration followed by subsequent administrations.

A method to potentially increase cell survival is to incorporate MHC-I negative cells, such as MAPCs, ES cells or other cells of interest into a biopolymer or synthetic polymer. Depending on the patient's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included cytokines, differentiation factors, angiogenesis factors and/or anti-apoptosis factors. Additionally, these could be in suspension. Another alternative is a three-dimension gel with cells entrapped within the interstices of the cell biopolymer admixture. Again cytokines, differentiation factors, angiogenesis factors and/or anti-apoptosis factors could be included within the gel. These could be deployed by injection via various routes described herein, via catheters or other surgical procedures.

In current human studies of autologous mononuclear bone marrow cells, empirical doses ranging from 1 to 4×10⁷ cells have been used. However, different scenarios may require optimization of the amount of cells injected into a tissue of interest. Thus, the quantity of cells to be administered will vary for the subject being treated. In a preferred embodiment, between 10⁴ to 10⁸, more preferably 10⁵ to 10⁷, and most preferably, 3×10⁷ MHC-I negative cells and optionally, 50 to 500 μg/kg per day of a cytokine can be administered to a human subject. However, the precise determination of what would be considered an effective dose may be based on factors individual to each patient, including their size, age, disease or injury, size damage, amount of time since the damage occurred and factors associated with the mode of delivery (direct injection—lower doses, intravenous—higher doses).

An issue regarding the use of stem cells is the purity of the population. Bone marrow cells, for example, comprise mixed populations of cells, which can be purified to a degree sufficient to produce a desired effect. Those skilled in the art can readily determine the percentage of MAPCs, ES cells or other MHC-I negative cells in a population using various well-known methods, such as fluorescence activated cell sorting (FACS). Preferable ranges of purity in populations comprising MHC-I negative cells, such as MAPCs or ES cells, or their differentiated progeny, are 50-55%, 55-60%, and 65-70%. More preferably the purity is 70-75%, 75-80%, 80-85%; and most preferably the purity is 85-90%, 90-95%, and 95-100%. However, populations with lower purity can also be useful, such as about <25%, 25-30%, 30-35%, 35-40%, 40-45% and 45-50%. Purity of, for example, MAPCs can be determined according to the gene expression profile within a population. Dosages can be readily adjusted by those skilled in the art (e.g., a decrease in purity may require an increase in dosage).

The skilled artisan can readily determine the amount of cells and optional additives, vehicles, and/or carrier in compositions and to be administered in methods of the invention. Typically, any additives (in addition to the active stem cell(s) and/or cytokine(s)) are present in an amount of 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response.

When administering a therapeutic composition of the present invention, it will generally be formulated in a unit dosage injectable form (solution, suspension, emulsion). The pharmaceutical formulations suitable for injection include sterile aqueous solutions and dispersions. The carrier can be a solvent or dispersing medium containing, for example, water, saline, phosphate buffered saline, polyol (for example, glycerol, propylene glycol, liquid polyethylene glycol, and the like) and suitable mixtures thereof.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells.

Sterile injectable solutions can be prepared by incorporating the cells utilized in practicing the present invention in the required amount of the appropriate solvent with various amounts of the other ingredients, as desired.

In one embodiment, MHC-I negative cells, such as MAPCs or ES cells, can be administered initially, and thereafter maintained by further administration of MHC-I negative cells, such as MAPCs or ES cells. For instance, MHC-I negative cells can be administered by one method of injection, and thereafter further administered by a different or the same type of method. For example, MAPCs or ES cells can be administered by surgical injection to bring lung function to a suitable level. The patient's levels can then be maintained, for example, by intravenous injection, although other forms of administration, dependent upon the patient's condition, can be used.

It is noted that human subjects are treated generally longer than the canines or other experimental animals, such that treatment has a length proportional to the length of the disease process and effectiveness. The doses may be single doses or multiple doses over a period of several days. Thus, one of skill in the art can scale up from animal experiments, e.g., rats, mice, canines and the like, to humans, by techniques from this disclosure and documents cited herein and the knowledge in the art, without undue experimentation. The treatment generally has a length proportional to the length of the disease process and drug effectiveness and the subject being treated.

Examples of compositions comprising MHC-I negative cells or differentiated progeny thereof, include liquid preparations for administration, including suspensions; and, preparations for direct or intravenous administration (e.g., injectable administration), such as sterile suspensions or emulsions. Such compositions may be in admixture with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the invention are conveniently provided as liquid preparations, e.g., isotonic aqueous solutions, suspensions, emulsions or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues.

The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Solutions, suspensions and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose), may also be present. The compositions can be isotonic, i.e., they can have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected and the desired viscosity. Viscous compositions are normally prepared from solutions by the addition of such thickening agents.

A pharmaceutically acceptable preservative or cell stabilizer can be employed to increase the life of the compositions. Preferably, if preservatives are necessary, it is well within the purview of the skilled artisan to select compositions that will not affect the viability or efficacy of the cells as described in the present invention.

Compositions can be administered in dosages and by techniques available to those skilled in the medical and veterinary arts taking into consideration such factors as the age, sex, weight, and condition of the particular patient, and the composition form used for administration (e.g., solid vs. liquid).

Suitable regimes for initial administration and further doses or for sequential administrations also are variable, may include an initial administration followed by subsequent administrations.

B. Additional Approaches for Transplantation to Prevent Immune Rejection

In some embodiments, it may be desired that the MHC-I negative cells, such as the MAPCs, ES cells or differentiated progeny thereof, be treated or otherwise altered prior to transplantation/administration in order to reduce the risk of stimulating host immunological response against the transplanted cells. Any method known in the art to reduce the risk of stimulating host immunological response may be employed. The following provides a few such examples.

1. Universal donor cells: MAPCs and ES cells have cell surface profiles consistent with evasion of immune recognition, and in their natural state may not stimulate immune sensitization and rejection. They may serve as natural universal donor cells even if their progeny mature to cells which ordinarily would be immune recognized and rejected.

Alternatively, MHC-I negative cells, such as MAPCs or ES cells, can be manipulated to serve as universal donor cells. Although undifferentiated MAPCs and ES cells do not express MHC-I or -II antigens, some differentiated progeny may express one or both of these antigens. MAPCs can be modified to serve as universal donor cells by eliminating MHC-I or MHC-II antigens, and potentially introducing the MHC-antigens from the prospective recipient so that the cells do not become easy targets for NK-mediated killing, or become susceptible to unlimited viral replication and/or malignant transformation. Elimination of MHC-antigens can be accomplished by homologous recombination or by introduction of point-mutations in the promoter region or by introduction of a point mutation in the initial exon of the antigen to introduce a stop-codon, such as with chimeroplasts. Transfer of the host MHC-antigen(s) can be achieved by retroviral, lentiviral, adeno associated virus or other viral transduction or by transfection of the target cells with the MHC-antigen cDNAs.

2. Intrauterine transplant to circumvent immune recognition: MAPCs or ES cells can be used in an intrauterine transplantation setting to correct genetic abnormalities, or to introduce cells that will be tolerated by the host prior to immune system development. This can be a way to make human cells in large quantities in animals or it could be used as a way to correct human embryo genetic defects by transplanting cells that make the correct protein or enzyme.

3. Immune Recognition and Tolerance:

A. Immune Recognition

Immune responses are controlled by molecular recognition events between receptors on T cells (T cell receptors or TCR) and somatic tissues (class I and II MHC). The TCR/MHC interactions are the antigen specific component of the immune response, enabling recognition between self and foreign antigen. While an immune reaction will only proceed following T cell recognition of a foreign or non-self antigen, additional signaling events are required and function to prevent accidental or autoimmune responses (Buckley, 2003).

Immune recognition can be divided into two phases, sensitization and secondary responses. Sensitization is accomplished by a subset of T cells, T helper cells, interacting with a specialized population of immune cells called dendritic cells. T helper cell recognition of antigen presented by class II MHC complexes on these dendritic or antigen-presenting cells (APC), is critical for initiating both antibody or cytolytic T cell responses. Only a limited number of cells express class II MHC receptors, and these “professional” APC are characterized by not only sensitizing T helper cells with non-self antigen, but also by expressing cytokine cascades that regulate amplification of T cells and control humoral versus cytolytic immune responses. B cells, macrophages, Langherhans cells, and other dendritic cell classes make up the APC compartment. Therefore, only specialized cell types can signal immune responsiveness, including allogeneic reactivity.

The two classes of MHC receptors, class I and II, have structural motifs that cause intracellular association with short peptide segments derived from all genes expressed in a cell. This complex of peptide bound to the MHC receptor on the cell surface is the molecular complex recognized by TCR, and therefore provides the specificity for antigenic recognition by T cells (analogous to a lock-and-key mechanism). Once the immune system has been sensitized and triggered, immune system cells amplify until the antigen is eliminated, and then reside in a resting or memory state to respond if the antigen is re-encountered.

Control of immune reactivity is accomplished in cascades. In addition to the primary recognition of non-self peptides between T helper cells and APC, a second stage is the required stimulation of APC by pathogen associated stimuli—for example, bacterial cell wall components such as LPS, viral particles that cross-link surface Ig on B cells; double-stranded RNA associated with viral infection; or inflammatory cytokines produced by physical wounding and damage to vasculature—all of these provide non-antigen specific confirmation that an immune response is warranted. The nature of these initial signals also triggers the APC to regulate humoral vs cellular responses by stimulating different cytokine cascades.

B. Tolerance

A second cascade that regulates the immune system is the restriction of the response to self-antigens by eliminating self-reactive T cells. For both B and T cell immunity, this is accomplished by regulating the repertoire of the T helper cell population, as this population determines reactivity in a sensitization reaction. T cells are produced in the bone marrow, and circulate to the thymus for “education” to distinguish between self and non-self antigens. T cells which can recognize self tissue are depleted during ontogeny in the thymus, to ensure that no T cells with T cell receptor complexes (TCR) reactive to self-antigen persist in circulation. This is termed central tolerance, and when broken, results in autoimmune disease.

A second type of tolerance can be induced, known as peripheral tolerance. This is accomplished when T cells that have passed through the thymus encounter non-self antigen, but do not receive secondary or co-stimulatory signals from APC that are required to trigger either helper or cytolytic function. This might occur when an APC has expressed antigen via a class II MHC receptor, but not received accessory signals as a consequence of infection or pathogen threat, and hence the APC does not express the cytokine cascade required for response. T cells partially stimulated in this fashion are rendered anergic or apoptotic. This results in depletion of the T helper population required for humoral or cytolytic responsiveness.

A second form of peripheral tolerance is generated when cytolytic T cells encounter cells expressing non-self antigen in class I MHC complexes on the majority of somatic cells. When the TCR of these T cells engage class I MHC in the absence of co-stimulatory receptor engagement (e.g., CD28/CD86 interaction), the T cells are rendered anergic or apoptotic. There is a panel of secondary co-stimulatory receptor interactions necessary and capable of providing this secondary signal, and therefore the surface phenotype of a cell can strongly predict immune stimulation or anergy.

Many tumor cells have evolved escape pathways from cytolytic recognition by down-regulating class I MHC expression, thus becoming invisible to the T cell arm of the immune system. Many viruses have evolved specific mechanisms for interfering with cell surface expression of MHC receptors in order to escape immune responses. An additional arm of the immune system has evolved to clear tumor cells, or virally infected cells with this property of reduced MHC expression. A population of cells termed natural killer or NK cells are capable of cytolytic activity against class I MHC negative cells. This activity is negatively regulated. NK cells bind target cells through interaction with receptors called Killer Inhibitory Receptors (KIR) and will kill unless turned off by interaction with class I MHC.

C. Hematopoietic Chimerism and Tolerance Induction

Bone marrow transplant is necessitated in cancer therapy where chemotherapeutic agents and/or radiation therapy results in myeloablation of the host immune system. The patient then reconstitutes immune function from the hematopoietic stem cells present in the bone marrow graft, and therefore has acquired the cellular and molecular components of the immune system from the bone marrow donor. The reconstitution of the donor immune system is accompanied by recapitulation of the self vs. non-self antigenic education seen in ontogeny, whereby the donor immune system is now tolerized to host tissues. A secondary aspect of donor immune system reconstitution is that the host is now capable of accepting an organ or tissue graft from the original donor without rejection.

When less severe myeloablative conditioning is used for bone marrow transplant, the host immune system may not be completely depleted, and with appropriate immunosuppressive management, a chimeric immune system may be reconstituted comprised of both donor and host immune cells. In this setting, the host is tolerized to the cellular and molecular components of both donor and host, and could accept an organ or tissue graft from the bone marrow donor without rejection. The clinical management of host rejection of donor bone marrow, and graft-versus-host response from donor bone marrow is the key to success in this therapeutic approach. The clinical risk of graft-versus-host response is a significant and as yet incompletely resolved risk in standardizing this approach for transplantation. These clinical protocols have received significant attention recently (Waldmann, 2004).

Significant benefit would be achieved through use of a stem cell, capable of reconstituting the immune system, that did not carry risk of graft-versus-host response. The graft-versus-host reaction is due to contaminating T cells inherent in the bone marrow graft. Although purification of hematopoietic stem cells from bone marrow is routine, their successful engraftment in the patient requires accompaniment by accessory T cells. Thus, a critical balance must be achieved between the beneficial engraftment value of T cells and the detrimental effect of graft-versus-host response.

MAPCs and ES cells represent a stem cell population which can be delivered without risk of graft-versus-host reactivity, as they can be expanded free of hematopoietic cell types including T cells. This greatly reduces clinical risk. The transient elimination of NK cell activity during the acute phase of cell delivery increases the frequency of primitive stem cell engraftment and hematopoietic reconstitution to a clinically useful threshold without risk of long term immunosuppression.

As MAPC or ES engraft and contribute to hematopoiesis, the newly formed T cells undergo thymic and peripheral self vs non-self education consistent with host T cells as described above. Co-exposure of newly created naïve T cells of donor and host origin results in reciprocal depletion of reactive cells, hence tolerance to T cells expression allogeneic antigens derived from a MAPC or ES donor can be achieved. A patient can thus be rendered tolerant to the cellular and molecular components of the MAPC or ES donor immune system, and would accept a cell, tissue or organ graft without rejection.

D. MAPC and Other Stem Cell Types

This above mechanism of tolerance induction is unique to a cell type capable of hematopoietic reconstitution. Although mesenchymal stem cells, also derived from bone marrow, have shown low immunogenicity and can persist in an allogeneic transplant setting, tolerance to donor immune components is not achieved. No other lineage committed stem cell has demonstrated hematopoietic reconstitution potential. This includes neuronal stem cells, fat-derived stem cells, liver stem cells, etc.

The ability to induce tolerance to subsequent graft acceptance using ES cells has been demonstrated by Fandrich (2002). In this setting, non-ablative conditioning accompanied by delivery of a murine ES cell type enabled animals to accept a heart allograft without rejection. Hence, the lineage regenerative properties common to ES cells and MAPC which includes hematopoietic reconstitution can achieve transplant tolerance. MAPCs represent an alternative to clinical use of ES cells for transplant tolerance.

Thus, the administration of MAPC or ES and the differentiation thereof into the various blood cell types can condition or prepare a recipient for secondary organ or tissue transplant with histocompatibility matching to the MAPC or ES cells. For example, a diabetic subject may be treated with cells obtained from, for example, a stem cell bank. Tolerization will follow and then one can provide to the diabetic subject allogeneic islet cells obtained or derived from the same source as the stem cell so that the mature islets are not rejected by the recipient. This process is available for any secondary transplant (e.g., organ, tissue and/or cell transplant) including, but not limited to, heart, liver, lung, kidney and/or pancreas.

4. Encapsulation: In some embodiments, the MHC-I negative cells, such as MAPCs or ES cells, are encapsulated. The primary goal in encapsulation as a cell therapy is to protect allogeneic and xenogeneic cell transplants from destruction by the host immune response, thereby eliminating or reducing the need for immuno-suppressive drug therapy. Techniques for microencapsulation of cells are known to those of skill in the art (see, for example, Chang, P., et al., 1999; Matthew, H. W., et al., 1991; Yanagi, K., et al., 1989; Cai Z. H., et al., 1988; Chang, T. M., 1992). Materials for microencapsulation of cells include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers. U.S. Pat. No. 5,639,275, for example, describes improved devices and methods for long-term, stable expression of a biologically active molecule using a biocompatible capsule containing genetically engineered cells.

Additionally, MHC-I negative cells may be encapsulated by membranes prior to implantation. The encapsulation provides a barrier to the host's immune system and inhibits graft rejection and inflammation. It is contemplated that any of the many methods of cell encapsulation available may be employed. In some instances, cells are individually encapsulated. In other instances, many cells are encapsulated within the same membrane. In embodiments in which the cells are removed following implantation, the relatively large size of a structure encapsulating many cells within a single membrane provides a convenient means for retrieval of the implanted cells. Several methods of cell encapsulation are available to the art, such as those described in European Patent Publication No. 301,777 or U.S. Pat. Nos. 4,353,888; 4,744,933; 4,749,620; 4,814,274; 5,084,350; 5,089,272; 5,578,442; 5,639,275; and 5,676,943, each of which is incorporated herein by reference.

C. Administration of Means for Inhibiting NK Cell Function

One or more means for inhibiting NK cell function, such as an anti-NK cell antibody or a compound (e.g., pharmaceutical, drug, small molecule or other chemical compound etc.), microorganism, protein, peptide, biologic, chemical, or nucleic acid (including vectors, such as expression vectors (e.g., the MHC-I negative cell, such as MAPCs or ES cells, can be genetically modified to produce an agent which inhibits the function of NK cells; this would result in inhibition of NK function in the vicinity of the transplanted MHC-I negative cell, and thus, the agent would not effect all NK cells of the recipient) can be formulated as a pharmaceutical composition. A pharmaceutical composition of the invention includes a means for inhibiting NK cell function in combination with a pharmaceutically acceptable carrier.

The means for inhibiting NK cell function can be administered by any suitable route, for example, orally, topically, or injected intravenously or intra-arterially, subcutaneously, intramuscularly, intraperitoneally, intrarectally, intravaginally, intranasally, intragastrically, intratracheally, or intrapulmonarily. The choice of the administration route depends on a number of parameters such as the nature of the means for inhibiting NK cell function and the disease or injury to be treated.

Administration of the means to inhibit NK cell function may take place in a single dose or in a dose repeated once or several times over a certain period. The appropriate dosage varies according to various parameters. Such parameters include the individual treated (adult or child), the means itself, the mode and frequency of administration, as will be determined by persons skilled in the art.

The pharmaceutical compositions of the invention may be prepared in many forms that include tablets, hard or soft gelatin capsules, aqueous solutions, suspensions, and liposomes and other slow-release formulations, such as shaped polymeric gels. Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An oral dosage form may be formulated such that means is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

A means for inhibiting NK cell function can be formulated for parenteral administration (e.g., by injection, for example, bolus injection or continuous infusion) and may be presented in unit dosage form in ampoules, pre-filled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, a means for inhibiting NK cell function may be in powder form, obtained by lyophilization from solution, for constitution with a suitable vehicle, e.g., sterile saline, before use.

Pharmaceutical compositions suitable for rectal administration can be prepared as unit dose suppositories. Suitable carriers include saline solution and other materials commonly used in the art.

For administration by inhalation, a means for inhibiting NK cell function can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, a means for inhibiting NK cell function may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, a means for inhibiting NK cell function may be administered via a liquid spray, such as via a plastic bottle atomizer.

Pharmaceutical compositions of the invention may also contain other ingredients such as flavorings, colorings, anti-microbial agents, anti-inflammatory agents or preservatives. It will be appreciated that the amount of a means for inhibiting NK cell function required for use in treatment will vary not only with the particular carrier selected but also with the route of administration, the severity of the disease or injury being treated and the age and condition of the patient. Ultimately the attendant health care provider may determine proper dosage.

Generally, a means for inhibiting NK cell function is generally administered in an amount sufficient to substantially deplete the subject's active Natural Killer cells, prevent the subject's Natural Killer cells from activating or otherwise inhibiting the activity of the subject's Natural Killer cells. The amount may vary dependent on the animal and type of means for inhibiting NK cell function selected. For example, although the effective dosage for each antibody must be titrated individually, most antibodies may be used in the dose range of 0.1 mg/kg-20 mg/kg body weight.

The administration of antibodies, or other means for inhibiting Natural Killer cells, can be performed prior to administering MHC-I negative cells to the subject, subsequent to administering the cells or after administering the cells to a subject. In one embodiment, administration of a means for inhibiting NK function can be performed sufficiently long before administration of MHC-I negative cells (for example, for a period of about 1-4 weeks) such that an advantageous alteration in the amounts of sub-populations or the activity/function of NK cells is obtained. In this manner, the beneficial effects of NK inhibition can be obtained prior to administering MHC-I negative cells, thereby reducing the probability of rejection of the transplanted cells.

When radiation is given in a way to cover the whole body it is called total body irradiation (TBI). Radiation can penetrate all areas of the body. This allows the treatment to reach cells even within scar tissue or deep recesses of the body. The radiation effect is generally on cells that are rapidly growing and/or have poor repair function. To take advantage of this, total body irradiation is generally given over several fractions, 2 to 3 times a day for 2 to 5 days.

The most sensitive cells in the body are the blood cells, which include lymphocytes, neutrophils, platelets, and red blood cells. Treatment with standard or high dose TBI as part of bone marrow transplant destroys these cells or their precursor stem cells, which then must be transfused back using stored bone marrow or blood stem cells obtained from the patient before treatment or from another person (donor). Low dose TBI is sometimes used to treat disorders of the blood cells such as low grade lymphoma and does not require bone marrow transplant or stem cells. Also contemplated within the present invention is low dose TBI, such as sub-lethal TBI, or localized irradiation (irradiation localized to a particular area or tissue within the body).

Other sensitive tissues include the lungs, GI tract, skin, liver, kidneys, and lens of the eye. In one embodiment, the methods of the invention are used to treat the damage caused by irradiation. However, partial blocking is sometimes used, depending on the TBI dose and disease being treated, to help prevent any lung damage. This blocking is prepared using special x-rays obtained at the time of treatment planning. To deliver total body irradiation in a homogenous manner, patient measurements are obtained and special “tissue compensators” may be required to make up for differences in body thickness.

Treatments are thus customized for the individual patient. They will take into account the equipment being used and the physical setup of the treatment room as well as the specific disease process and patient characteristics (size, thickness, and lung volumes). Because children are actively growing, their normal tissues are often more sensitive to radiation, and the toxicity of TBI treatment can be different for them; it may even vary with the age of the child.

Also, various pharmaceuticals and nonmyeloablative protocols which can be used in place of, or in combination with, TBI are within the scope of the present invention.

In one embodiment, bone marrow is administered in combination with TBI and MHC-I negative cell administration, and optionally administration of an additional means for inhibiting NK cell function or other agents to suppress or inhibit immune function. Bone marrow transplantation (BMT) is generally therapy for patients with cancer or other diseases which affect the bone marrow. A bone marrow transplant involves taking cells that are normally found in the bone marrow, such as hematopoietic or blood-forming stem cells, filtering those cells, and giving them back either to the patient or to another person. The goal of BMT is to transfuse healthy bone marrow cells into a person after their own unhealthy bone marrow has been eliminated. Peripheral blood stem cell transplantation (PBSCT) is another method of replacing blood-forming cells destroyed by medicinal treatments and/or disease (e.g., immature blood cells in the circulating blood, that are similar to those in the bone marrow, are given to the patient after treatment to aid in the recovery of the bone marrow and to continue producing healthy blood cells). Included herewith are mini-transplants (use of lower, less toxic doses of chemotherapy and/or radiation to prepare the patient for transplant) and tandem transplants (use of two sequential courses of high-dose chemotherapy and cell transplant).

Diseases and/or disorders that may be treated with BMT or PBSCT include but are not limited to leukemia, lymphomas, multiple myeloma, solid tumors (including neuroblastoma, rhabdomyosarcoma and/or brain tumors), aplastic anemia (Fanconi anemia (FA) is one of the inherited anemias that leads to bone marrow failure (aplastic anemia)), immune deficiencies (including severe combined immunodeficiency disorder, or Wiskott-Aldrich Syndrome), sickle cell disease, thalassemia, Blackfan-Diamond anemia, metabolic/storage diseases (including Hurler's syndrome or adrenoleukodystrophy disorder), and cancers of the breast, ovaries, and kidneys.

D. Monitoring of Subject after Administration of MHC-I Negative Cells

Following transplantation, the growth and/or differentiation of the administered MHC-I negative cells or differentiated progeny, and the therapeutic effect of the MHC-I negative cells or progeny may be monitored. For example, the functionality of MHC-I negative cells administered to treat a pancreatic disease may be monitored by analyzing serum glucose levels. Normalization of serum glucose levels in the serum of a diabetic subject following administration of MHC-I negative cells is indicative of functionality.

The functionality of MHC-I negative cells to treat a cardiac disease may be monitored by various well-known techniques such as scintigraphy, myocardial perfusion imaging, gated cardiac blood-pool imaging, first-pass ventriculography, right-to-left shunt detection, positron emission tomography, single photon emission computed tomography, magnetic resonance imaging, harmonic phase magnetic resonance imaging, echocardiography, electrocardiography, analysis of cardiac function-specific proteins in the serum of the subject and myocardial perfusion reserve imaging.

Following administration, the immunological tolerance of the subject to the MHC-I negative cells or progeny derived therefrom may be tested by various methods known in the art to assess the subject's immunological tolerance to MHC-I negative cells or progeny derived therefrom. In cases where subject tolerance of MHC-I negative cells or progeny derived therefrom is suboptimal (e.g., the subject's immune system is rejecting the exogenous MAPCs), therapeutic adjunct immunosuppressive treatment, which is known in the art, of the subject may be performed.

### Genetically-Modified MHC-I Negative Cells

MHC-I negative cells, such as MAPCs or ES cells, or their differentiated progeny can be genetically altered ex vivo, eliminating one of the most significant barriers for gene therapy. For example, a subject's bone marrow aspirate is obtained, and from the aspirate MAPCs are isolated. The MAPCs are then genetically altered to express one or more desired gene products. The MAPCs can then be screened or selected ex vivo to identify those cells which have been successfully altered, and these cells can be introduced into the subject or can be differentiated and introduced into the subject, either locally or systemically. Alternately, MHC-I negative cells, such as MAPCs or ES cells, can be differentiated and then the differentiated cells can be genetically altered prior to administration.

In either case, the transplanted cells provide a stably-transfected source of cells that can express a desired gene product. Genetically-modified MHC-I negative cells, such as MAPCs or ES cells, or their genetically-modified differentiated progeny are useful in the methods of the invention, for example, in the treatment of genetic disorders, including but not limited to mucoviscidosis (cystic fibrosis) and immotile cilia syndrome, or to provide a gene product to a desired tissue (e.g., lung tissue).

A. Methods for Genetically Altering MHC-I Negative Cells

MHC-I negative cells, such as MAPCs or ES cells, can be genetically modified by introducing DNA or RNA (e.g., an exogenous nucleic acid) into the cell by a variety of methods known to those of skill in the art. These methods are generally grouped into four major categories: (1) viral transfer, including the use of DNA or RNA viral vectors, such as retroviruses (including lentiviruses), Simian virus 40 (SV40), adenovirus, Sindbis virus, and bovine papillomavirus for example; (2) chemical transfer, including calcium phosphate transfection and DEAE dextran transfection methods; (3) membrane fusion transfer, using DNA-loaded membranous vesicles such as liposomes, red blood cell ghosts, and protoplasts, for example; and (4) physical transfer techniques, such as microinjection, electroporation, nucleofection, or direct “naked” DNA transfer. Cells can be genetically altered by insertion of pre-selected isolated DNA, by substitution of a segment of the cellular genome with pre-selected isolated DNA, or by deletion of or inactivation of at least a portion of the cellular genome of the cell. Deletion or inactivation of at least a portion of the cellular genome can be accomplished by a variety of means, including but not limited to genetic recombination, by antisense technology (which can include the use of peptide nucleic acids, or PNAs), or by ribozyme technology, for example. Insertion of one or more pre-selected DNA sequences can be accomplished by homologous recombination or by viral integration into the host cell genome. Methods of non-homologous recombination are also known, for example, as described in U.S. Pat. Nos. 6,623,958, 6,602,686, 6,541,221, 6,524,824, 6,524,818, 6,410,266, 6,361,972, the contents of which are specifically incorporated by reference for their entire disclosure relating to methods of non-homologous recombination.

The desired gene sequence can also be incorporated into the cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals (including but not limited to the tamoxifen responsive mutated estrogen receptor) expression in specific cell compartments (including but not limited to the cell membrane).

Calcium phosphate transfection, which relies on precipitates of plasmid DNA/calcium ions, can be used to introduce plasmid DNA containing a target gene or polynucleotide into isolated or cultured MHC-I negative cells. Briefly, plasmid DNA is mixed into a solution of calcium chloride, and then added to a solution which has been phosphate-buffered. Once a precipitate has formed, the solution is added directly to cultured cells. Treatment with DMSO or glycerol can be used to improve transfection efficiency, and levels of stable transfectants can be improved using bis-hydroxyethylamino ethanesulfonate (BES). Calcium phosphate transfection systems are commercially available (e.g., ProFection® from Promega Corp., Madison, Wis.).

DEAE-dextran transfection, which is also known to those of skill in the art, may be preferred over calcium phosphate transfection where transient transfection is desired, as it is often more efficient.

Microinjection can be particularly effective for transferring genetic material into the cells. Briefly, cells are placed onto the stage of a light microscope. With the aid of the magnification provided by the microscope, a glass micropipette is guided into the nucleus to inject DNA or RNA. This method is advantageous because it provides delivery of the desired genetic material directly to the nucleus, avoiding both cytoplasmic and lysosomal degradation of the injected polynucleotide. This technique has been used effectively to accomplish germline modification in transgenic animals.

Cells can also be genetically modified using electroporation. The target DNA or RNA is added to a suspension of cultured cells. The DNA/RNA-cell suspension is placed between two electrodes and subjected to an electrical pulse, causing a transient permeability in the cell's outer membrane that is manifested by the appearance of pores across the membrane. The target polynucleotide enters the cell through the open pores in the membrane, and when the electric field is discontinued, the pores close in approximately one to 30 minutes.

Liposomal delivery of DNA or RNA to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPC) can be added. A recommended reagent for liposomal transfer is Lipofectin® (Life Technologies, Inc.), which is commercially available. Lipofectin®, for example, is a mixture of the cationic lipid N-[1-(2,3-dioleyloyx)propyl]-N—N—N-trimethyl ammonia chloride and DOPE. Delivery of linear DNA, plasmid DNA, or RNA can be accomplished either in vitro or in vivo using liposomal delivery, which may be a preferred method due to the fact that liposomes can carry larger pieces of DNA, can generally protect the polynucleotide from degradation, and can be targeted to specific cells or tissues. A number of other delivery systems relying on liposomal technologies are also commercially available, including Effectene™ (Qiagen), DOTAP (Roche Molecular Biochemicals), FuGene 6™ (Roche Molecular Biochemicals), and Transfectam® (Promega). Cationic lipid-mediated gene transfer efficiency can be enhanced by incorporating purified viral or cellular envelope components, such as the purified G glycoprotein of the vesicular stomatitis virus envelope (VSV-G), in the method of Abe, A., et al., 1998).

Gene transfer techniques which have been shown effective for delivery of DNA into primary and established mammalian cell lines using lipopolyamine-coated DNA can be used to introduce target DNA into MHC-I negative cells of the invention. This technique is generally described by Loeffler, J. and Behr, J., 1993).

Naked plasmid DNA can be injected directly into a tissue mass formed of differentiated cells, such as the vascular endothelial cells of the invention. This technique has been shown to be effective in transferring plasmid DNA to skeletal muscle tissue, where expression in mouse skeletal muscle has been observed for more than 19 months following a single intramuscular injection. More rapidly dividing cells take up naked plasmid DNA more efficiently. Therefore, it is advantageous to stimulate cell division prior to treatment with plasmid DNA.

Microprojectile gene transfer can also be used to transfer genes into cells either in vitro or in vivo. The basic procedure for microprojectile gene transfer was described by J. Wolff in “Gene Therapeutics” (1994) at page 195. Briefly, plasmid DNA encoding a target gene is coated onto microbeads, usually 1-3 micron sized gold or tungsten particles. The coated particles are placed onto a carrier sheet inserted above a discharge chamber. Once discharged, the carrier sheet is accelerated toward a retaining screen. The retaining screen forms a barrier which stops further movement of the carrier sheet while allowing the polynucleotide-coated particles to be propelled, usually by a helium stream, toward a target surface, such as a tissue mass formed of differentiated MAPCs. Microparticle injection techniques have been described previously, and methods are known to those of skill in the art (see Johnston, S. A., et al., 1993; Williams, R. S., et al., 1991; Yang, N. S., et al., 1990.

Signal peptides can be attached to plasmid DNA, as described by Sebestyen, et al. (1998), to direct the DNA to the nucleus for more efficient expression.

Viral vectors can be used to genetically alter MHC-I negative cells and their progeny. Viral vectors are used, as are the physical methods previously described, to deliver one or more target genes, polynucleotides, antisense molecules, or ribozyme sequences, for example, into the cells. Viral vectors and methods for using them to deliver DNA to cells are well known to those of skill in the art. Examples of viral vectors which can be used to genetically alter the cells of the present invention include, but are not limited to, adenoviral vectors, adeno-associated viral vectors, retroviral vectors (including lentiviral vectors), alphaviral vectors (e.g., Sindbis vectors), and herpes virus vectors.

Retroviral vectors are effective for transducing rapidly-dividing cells, although a number of retroviral vectors have been developed to effectively transfer DNA into non-dividing cells as well (Mochizuki, H., et al., 1998. Packaging cell lines for retroviral vectors are known to those of skill in the art. Packaging cell lines provide the viral proteins needed for capsid production and virion maturation of the viral vector. Generally, these include the gag, pol, and env retroviral genes. An appropriate packaging cell line is chosen from among the known cell lines to produce a retroviral vector which is ecotropic, xenotropic, or amphotropic, providing a degree of specificity for retroviral vector systems.

A retroviral DNA vector is generally used with the packaging cell line to produce the desired target sequence/vector combination within the cells. Briefly, a retroviral DNA vector is a plasmid DNA which contains two retroviral LTRs positioned about a multicloning site and SV40 promoter so that a first LTR is located 5 to the SV40 promoter, which is operationally linked to the target gene sequence cloned into the multicloning site, followed by a 3′ second LTR. Once formed, the retroviral DNA vector can be transferred into the packaging cell line using calcium phosphate-mediated transfection, as previously described. Following approximately 48 hours of virus production, the viral vector, now containing the target gene sequence, is harvested.

Targeting of retroviral vectors to specific cell types was demonstrated by Martin, F., et al. (1999), who used single-chain variable fragment antibody directed against the surface glycoprotein high-molecular-weight melanoma-associated antigen fused to the amphotropic murine leukemia virus envelope to target the vector to delivery the target gene to melanoma cells. Where targeted delivery is desired, as, for example, when differentiated cells are the desired objects for genetic alteration, retroviral vectors fused to antibody fragments directed to the specific markers expressed by each cell lineage differentiated from, for example, MAPCs or ES cells, can be used to target delivery to those cells.

Lentiviral vectors are also used to genetically alter MHC-I negative cells. Many such vectors have been described in the literature and are known to those of skill in the art. (Salmons, B. and Gunzburg, W. H., 1993). These vectors have been effective for genetically altering human hematopoietic stem cells (Sutton, R., et al., 1998). Packaging cell lines have been described for lentivirus vectors (see Kafri, T., et al., 1999; Dull, T., et al., 1998).

Recombinant herpes viruses, such as herpes simplex virus type I (HSV-1) have been used successfully to target DNA delivery to cells expressing the erythropoietin receptor (Laquerre, S., et al., 1998). These vectors can also be used to genetically alter MHC-I negative cells.

Adenoviral vectors have high transduction efficiency, can incorporate DNA inserts up to 8 Kb, and can infect both replicating and differentiated cells. A number of adenoviral vectors have been described in the literature and are known to those of skill in the art (see, for example, Davidson, B. L., et al., 1993; Wagner, E., et al., 1992). Methods for inserting target DNA into an adenovirus vector are known to those of skill in the art of gene therapy, as are methods for using recombinant adenoviral vectors to introduce target DNA into specific cell types (see Wold, W., Adenovirus Methods and Protocols, Humana Methods in Molecular Medicine (1998), Blackwell Science, Ltd.). Binding affinity for certain cell types has been demonstrated by modification of the viral vector fiber sequence. Adenovirus vector systems have been described which permit regulated protein expression in gene transfer (Molin, M., et al., 1998). A system has also been described for propagating adenoviral vectors with genetically modified receptor specificities to provide transductional targeting to specific cell types (Douglas, J., et al., 1999). Recently described ovine adenovirus vectors even address the potential for interference with successful gene transfer by preexisting humoral immunity (Hofmann, C., et al., 1999).

Adenovirus vectors are also available which provide targeted gene transfer and stable gene expression using molecular conjugate vectors, constructed by condensing plasmid DNA containing the target gene with polylysine, with the polylysine linked to a replication-incompetent adenovirus (Schwarzenberger, P., et al., 1997).

Alphavirus vectors, particularly the Sindbis virus vectors, are also available for transducing the cells of the present invention. These vectors are commercially available (Invitrogen, Carlsbad, Calif.) and have been described in, for example, U.S. Pat. No. 5,843,723, as well as by Xiong, C., et al., 1989; Bredenbeek, P. J., et al., 1993; and Frolov, I., et al., 1996).

Additionally, MAPCs possess good transduction potential using the eGFP-MND lentiviral vector described by Robbins, et al. (1997) and eGFP-MGF vector. Using this method, 30-50% of MAPCs, or any MHC-I negative cell, can be transduced after a short exposure of 4.6 hours to an enhanced green fluorescent protein (eGFP) vector containing supernatants made in PA3-17 packaging cells (an amphotropic packaging cell line derived from NIH 3T3 fibroblasts and described by Miller, A. D., and C. Buttimore (1986), combined with protamine (8 mg/ml). Expression of eGFP persists throughout the culture of undifferentiated MAPC. In addition, transfection using lipofectamine has been successfully used to introduce transgenes in MAPCs and can be use to introduce transgenes into any MHC-I negative cell.

Successful transfection or transduction of target cells can be demonstrated using genetic markers, in a technique that is known to those of skill in the art. The green fluorescent protein of Aequorea victoria, for example, has been shown to be an effective marker for identifying and tracking genetically modified hematopoietic cells (Persons, D., et al., 1998). Alternative selectable markers include the β-Gal gene, the truncated nerve growth factor receptor, drug selectable markers (including but not limited to NEO, MTX, hygromycin).

Any of these techniques can also be applied to introduce a transcriptional regulatory sequence into MHC-I negative cells to activate a desired endogenous gene. This can be done by both homologous (e.g., U.S. Pat. No. 5,641,670) or non-homologous (e.g., U.S. Pat. No. 6,602,686) recombination. These are incorporated by reference for teaching of general methods of homologous or non-homologous recombination and specifically endogenous gene activation.

### EXAMPLE

The following example is provided in order to demonstrate and further illustrate certain embodiments and aspects of the present invention and is not to be construed as limiting the scope thereof.

### Example 1

### Materials and Methods

Mouse Strains

C57BL/6 and recombinase activating gene-2 deficient (Rag2^{-/-}) mice were obtained from The Jackson Laboratory (Bar Harbor, Me.) and Taconic Farms (Germantown, N.Y.), respectively. Mice carrying mutations in the recombinase activating gene 2 and the common cytokine receptor (Rag2^{-/-}-IL-2Rγc^{-/-}) were a gift from Dr. Stephen Jameson (University of Minnesota). All mice were housed under specific-pathogen free conditions, fed ad libitum according to University of Minnesota Research Animal Resources guidelines, and used at 6-12 weeks of age.

NK Depletion

To deplete NK cells in vivo, some mice were injected with anti-NK1.1 monoclonal antibody (hydridoma PK136, rat IgG₂ₐ; provided by Dr. Koo, Rahway, N.J.) 3 days before MAPC infusion and then twice a week for 30 days.

Bone Marrow Transplantation

B10.BR mice (H2^{d}) were lethally irradiated with 8.0 Gy by x-ray on the day prior to transplantation of with 20×10⁶ C57BL/6 (H2^{b}) bone marrow cells with or without 10⁶ MAPC DL (H2^{b}).

MAPC Culture, Labeling and Injection

MAPCs were isolated from adult C57BL/6J-rosa26 (H2^{b}, transgenic for lacZ and NeoR genes) bone marrow, cultured at low density in fibronectin (Sigma Chemical Corporation, St Louis, Mo.) coated flasks, and induced to differentiate in vitro into neurons, hepatocytes and endothelium as described previously (Jiang et al., 2002a). A single MAPC-derived clone stably expressing DsRed2 and firefly luciferase was prepared using Sleeping Beauty transposons (Ivics et al., 1997).

For intravenous injections, MAPCs were infused via the tail vein. Intra-arterial injections were performed as follows: Under general anesthesia, small midline upper abdominal incision was performed and the caudal aspect of diaphragm was exposed. After direct visualization of heart apex, 10⁶ MAPC (in 10 microliters of PBS) was slowly injected across the diaphragmatic and left ventricular wall.

Flow Cytometry

Single cell suspensions of MAPCs were prepared in buffer (PBS+2% bovine serum+0.15% sodium azide). Pelleted cells were incubated for 15 minutes at 4° C. with 0.4 μg of anti-Fc receptor monoclonal antibody (mAb; clone 2.4G2, rat IgG_{2b}) to prevent Fc binding. Flow cytometry using directly conjugated (fluorescein isothiocyanate, FITC, or phycoerythrin, PE) mAbs was performed to assess cell surface antigen expression of MAPCs before and after 24 hour stimulation with 1,000 units of IFNγ/mL (R&D Systems Inc., Minneapolis, Minn.). Optimal concentrations of directly conjugated mAbs were added to a total volume of 100 to 130 μL and incubated for 1 hour at 4° C. The mAbs obtained from Pharmingen (San Diego, Calif.) included: anti-H2^{b} specific mAb (clone EH-144, mouse IgG₂ₐ), anti-IA^{b} specific mAb (clone AF6-120.1, mouse IgG₂ₐ), anti-CD80 specific mAb (clone 16-10A1, Hamster IgG₂), anti-CD86 specific mAb (clone GL1, rat IgG₂ₐ), anti-ICAM-1 specific mAb (clone 3E2, Hamster IgG₁), and anti-CD40 specific mAb (clone HM40-3, Hamster IgMₖ). All samples were analyzed on a FACScalibur (Becton Dickinson, Palo Alto, Calif.) using Cell Quest software. Forward and 90 degree side-scatter were used to identify and gate live MAPC population. A minimum of 10,000 events was examined.

Mixed Lymphocyte Reaction (MLR) Culture

To measure the potential of MAPCs to stimulate allogeneic T cell responses, purified CD4⁺ T cells or whole T cells were prepared from single cell suspensions of axillary, inguinal, and mesenteric lymph node cells isolated from BALB/c mice. Lymph node cells were depleted of natural killer (NK) cells for all cell preparations and CD8⁺ T cells (hybridoma 2.43, rat IgG_{2b}; provided by Dr. Sachs, Charlestown, Mass.) for CD4⁺ cell preparations by coating with monoclonal antibodies and passage through a goat anti-rat-Ig-coated column (Cedarlane Laboratories, Hornby, ON, Canada). 10⁵ purified T cells and 10³ irradiated (3000 cGy by ¹³⁷Cs irradiation) MAPCs per well were plated in 96-well round bottom plates at 37° C. and 10% CO₂ for 5 days in DMEM (BioWhittaker, Walkersville, Md.) containing 10% FCS (Hyclone, Logan, Utah), 50 mM 2-mercaptoethanol (2-ME; Sigma), 10 mM HEPES (N-2-hydroxyethylpiperazine-N′-2-ethanesulfonic acid) buffer, 1 mM sodium pyruvate (Life Technologies, Grand Island, N.Y.), amino acid supplements (1.5 mM L-glutamine, L-arginine L-asparagine) (Sigma), and antibiotics (100 U/mL penicillin; 100 mg/mL streptomycin) (Sigma). Some MAPCs were pretreated with 1,000 IU of IFN-γ for 48 hours before initiating assay. Irradiated, T cell depleted splenocytes were prepared from C57BL/6 mice as a positive control for T cell proliferation. On day 5 of culture, each well was pulsed with tritiated thymidine (1 μCi/well) (Amersham Life Sciences, Buckinghamshire, United Kingdom) for 18 hours prior to harvesting and counted in the absence of scintillation fluid on a β-plate reader (Packard Instrument Company, Meriden, Conn.). Four wells were analyzed per group.

NK Lysis

To induce NK activity in effector cells, C57BL/6 mice were injected intraperitoneally with poly I:C (120 μg/mouse), and after 48 hours splenocytes were harvested. Target cells (MAPCs or Yac-1 cells) were loaded with ⁵¹Cr 1 hour before the experiment and washed three times as described previously (Kim et al., 2002). In 96-well plates, labelled MAPCs or Yac-1 cells (about 5,000/well) were mixed with splenocytes from poly I:C injected mice at various ratios (200:1 to 0.8:1). Cells were incubated at room temperature for 4 hours and harvested by centrifugation (5 minutes at 500 rpm). To test cytolytic potential of cells, γ radioactivity was measured in the supernatant and expressed as counts per minute (cpm). A total of three assays were performed. Relative target cell lysis was calculated as: (sample cpm−spontaneous release)/(maximum release-spontaneous release)×100%. Spontaneous release was less than 1.5%.

In Vivo Imaging of MAPCs

At 30 days after MAPC infusion, experimental mice were anesthetized with Nembutol (0.1 cc/10 mg body weight) and the abdomen and chest were shaved. Luciferin stock (30 mg/ml, Xenogen, Alameda, Calif.) was injected into the mice at 150 mg/kg intraperitoneally. A grayscale reference image was taken of the position of the mice prior to assessing luciferase activity. Bioluminescent signals were assessed at 5 min post luciferin injection at an integration time of 2 minutes using an in vivo imaging system that utilizes a cooled charge-coupled device (CCD) camera (IVIS100, Xenogen). Pseudocolor images representing the bioluminescent signal intensity (blue is the least intense and red is the most intense) were superimposed over the grayscale reference image. The scales for the pseudocolor intensity plots were displayed with the images.

In Vitro Quantification of Luciferase Expression

Tissue homogenates of lung specimens were harvested by centrifugation, mixed with 10 μL of luciferin stock (30 mg/mL, Xenogen), and assayed immediately for bioluminescence activity on a Chameleon 425-100 Multi-label Counter (Hidex, Turku, Finland). Average relative luminescence values were expressed as counts/second and normalized to total protein (Dojindo Molecular Technologies, Gaithersburg, Md.).

Tissue Immunohistochemistry for MAPC Localization and Differentiation

Tissue specimens of the recipient animals were cryopreserved in optimal cutting temperature (OCT) medium (Sakura Finetek, Torrence, Calif.) at −80° C. Six micrometer thick fresh frozen sections were mounted on glass slides, fixed in acetone for 10 min at room temperature and incubated in isotype sera for 20 min. Cryosections were stained with nuclear stain 4′,6-diamidino-2-phenylindole, DAPI (Molecular Probes, Eugene, Oreg.) and examined for native fluorescence of DsRed2 by confocal fluorescence microscopy (Olympus AX70, Olympus optical Co. LTD, Japan). To assess histological location of alveolar type I pneumocytes, lung sections were also stained with primary rabbit anti-aquaporin 5 antibody at 1:250 (Chemicon International, Temecula, Calif.) and incubated 1 hour at room temperature. Slides were washed twice in PBS and secondary donkey anti-rabbit IgG (Jackson ImmunoResearch, West Grove, Pa.) was added at 1:1000 and incubated for 1 hour at room temperature. Slides were examined using confocal fluorescence microscopy.

### Results

MAPCs as Targets of T Cell and NK Cell Immune Response as Assessed In Vitro

A. Flow Cytometry Analysis

Immunophenotyping revealed that MAPCs were low/negative for MHC class I and class II, costimulatory moleculars (CD80, CD86, CD40) and the adhesions molecule ICAM-1 (C54) (Table 1). Upon 24 hour stimulation with interferon γ (IFN-γ), MHC class I and ICAM-1 expression was upregulated, while the expression of MHC class II, CD80, CD86, and CD40 remained low. Because of the low/negative MHC class I expression, MAPCs may be good targets for NK mediated elimination.

TABLE 1
Flow Cytometry Analysis of MAPCs.

| IFN-γ | Antigen | Mean | SD | P-value |
|---|---|---|---|---|
| no | H2^{b} | 9.3 | 0.5 | |
| yes | | 99.0 | 1.4 | 0.00 |
| no | Ia^{b} | 4.5 | 2.1 | |
| yes | | 4.0 | 1.1 | 0.78 |
| no | CD80 | 10.7 | 2.0 | |
| yes | | 7.9 | 1.0 | 0.17 |
| no | CD86 | 0.1 | 0.1 | |
| yes | | 0.1 | 0.0 | 0.40 |
| no | ICAM-1 | 2.9 | 1.6 | |
| yes | | 35.9 | 10.7 | 0.01 |
| no | CD40 | 2.0 | 1.2 | |
| yes | | 1.8 | 1.0 | 0.86 |

Values are expressed as percent (%) of total cells gated.
IU, international unit;
h, hour, SD, standard deviation.

B. MAPCs do not Stimulate T Cell Responses In Vitro

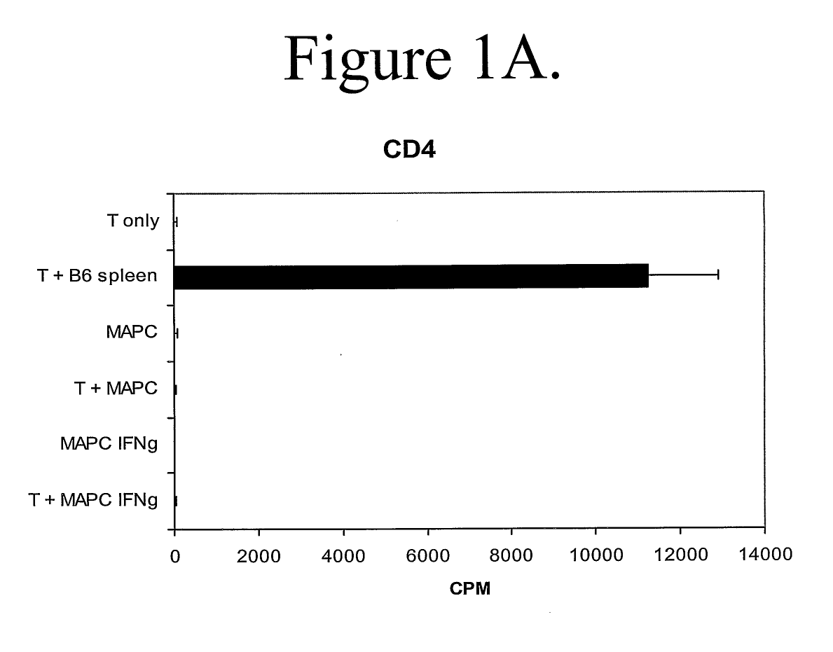

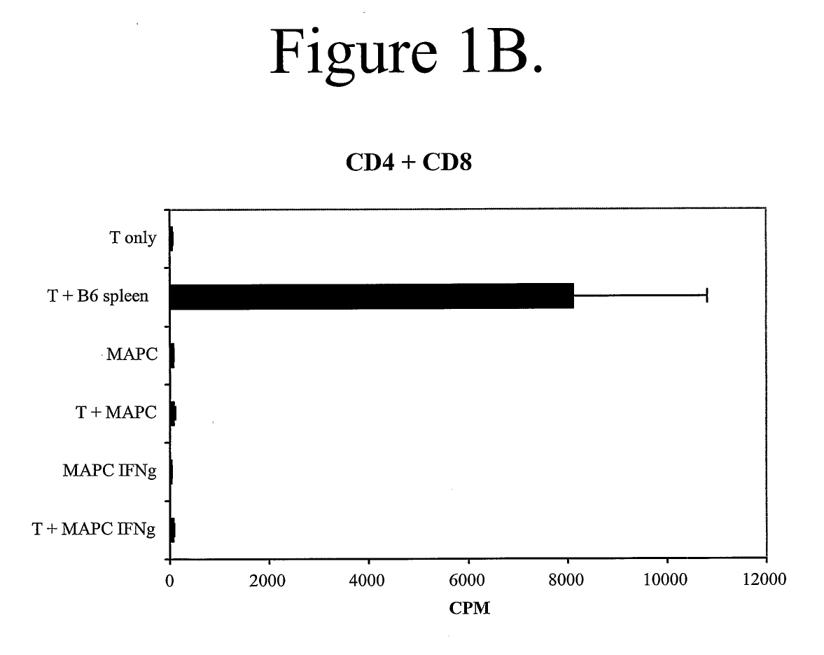

In order to determine whether MAPCs can stimulate T cells, allogeneic T cell proliferation assays were performed using BALB/c CD4⁺ T cells or BALB/c CD4⁺ plus CD8⁺ T cells (H2^{d}) as responders and C57BL/6 MAPC(H2^{b}) as stimulators. Neither untreated MAPCs nor MAPCs pretreated with IFN-γ for 48 hours to upregulate MHC Class I expression (data not shown, and Table 1) stimulated CD4⁺ only (FIG. 1A) or whole T cell alloresponses (FIG. 1B) in vitro.

C. MAPCs are Susceptible to NK Mediated Lysis In Vitro

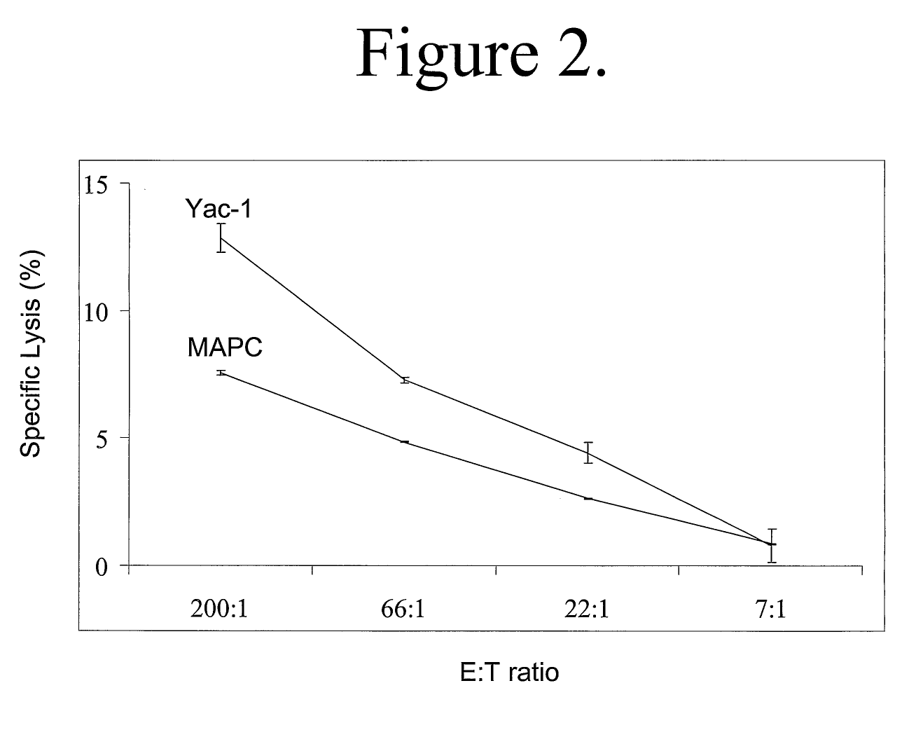

Splenocytes from poly I:C (an inducer of NK activity) treated C57BL/6 mice were mixed with an NK sensitive target, Yac-1 (H2^{a}), an NK sensitive target, or MAPCs in a 4 hour chromium release assay. Effector to target ratios indicated that MAPCs were susceptible to NK lysis, but less so than Yac-1 cells (FIG. 2).

Quantification of In Vivo Immune Resistance to MAPC in Real Time

To assess in vivo immune responses to MAPCs, MAPCs were infused into mice with various degrees of immune competence. For labeling, MAPCs were nucleoporated with Sleeping Beauty transposon constructs to drive expression of DsRed2 and firefly luciferase to yield a doubly transgenic MAPC DL (DsRed2, luciferase). To sequentially follow homing, migration and persistence of MAPCs in live animals in vivo, whole body imaging (WBI) was performed using the luciferase-mediated bioluminescent imaging (BLI). One million MAPC DL were injected intravenously into adult C57BL/6 or T- and B-cell deficient Rag2^{-/-} mice. Additional cohorts of C57BL/6 or Rag2^{-/-} mice were given anti-NK1.1 monoclonal antibody to deplete NK cells (administered three days before MAPC infusion and then twice a week thereafter). These data were compared to Rag2^{-/-}/IL-2Rγc^{-/-} mice that lack T-, B-, and NK-cells in sequential BLI analysis on days 4, 14 and 30 after MAPC DL infusion.

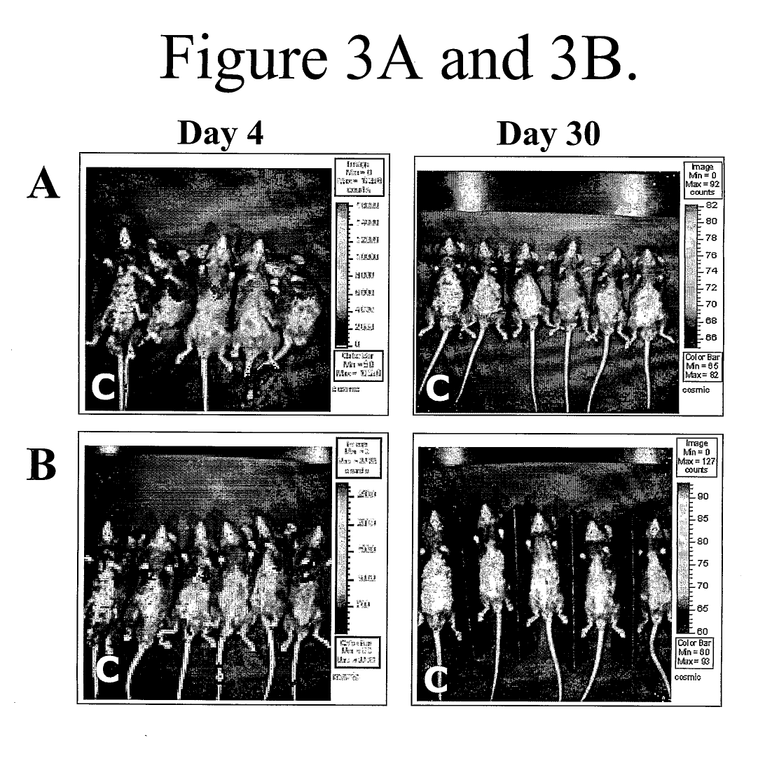

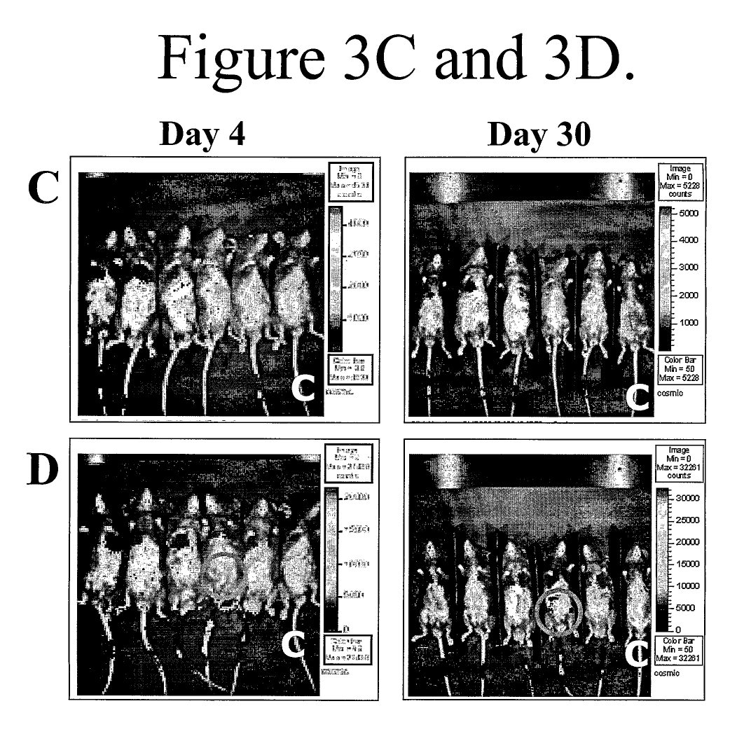

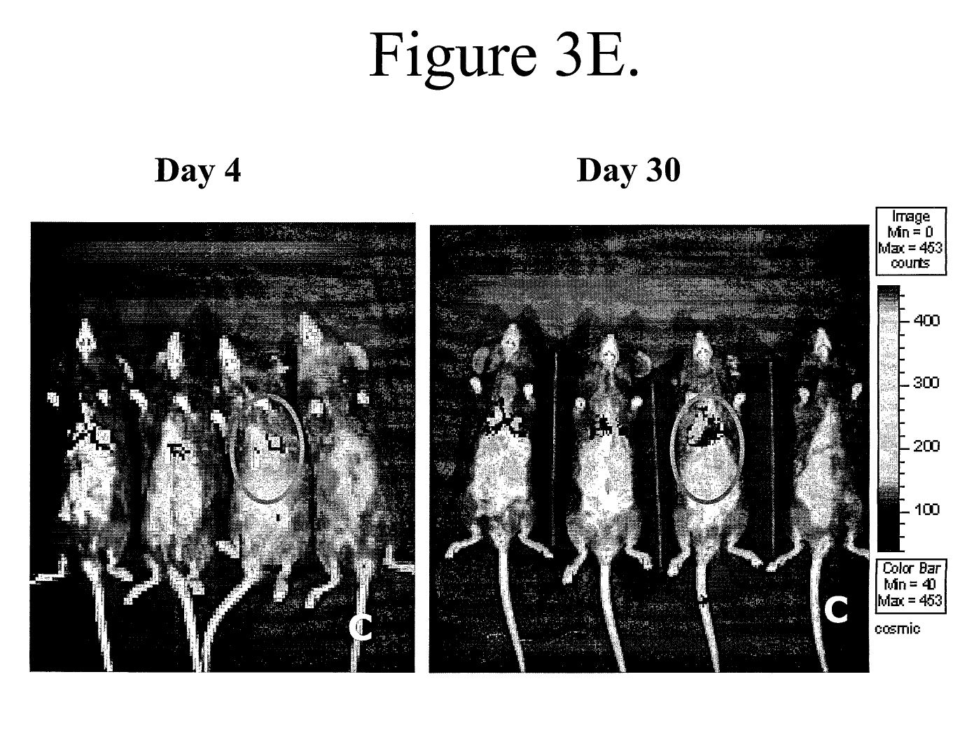

In C57BL6 mice, MAPC DL were detected in the lung and the injection site (tail vein) on day 4, but not day 14 or day 30 (FIG. 3A). In Rag2^{-/-} mice, MAPC DL were detected throughout the 30 day period (FIG. 3C). While NK depletion did not substantially increase MAPC DL number by BLI quantification in B6 mice, it did in Rag2^{-/-} by day 30 (FIGS. 3B and 3D).

In Rag2^{-/-}/IL-2Rγc^{-/-} mice, MAPC DL were persistent and in about 50% of mice increased in number from day 4 to day 30 (FIG. 3E). Collectively, these data suggest that endogenous NK cells resist MAPC DL. In B6 mice, depletion of NK cells was insufficient to overcome MAPC DL resistance. Rag2^{-/-} mice, which have higher NK activity than B6 mice (Prlic et al, 2001), showed low level of MAPC DL engraftment, unless they were in vivo depleted of NK cells (Table 2).

Thus, NK cells resist MCH I low/negative MAPCs. As NK depletion alone resulted in no engraftment in T and B cell competent mice and in low levels of engraftment in mice with depleted T- and B-cell function and intact NK cells (FIG. 3, Table 2), the data presented herein also indicate that T- (or B-) cells play a role in immune resistance to MAPC engraftment in vivo.

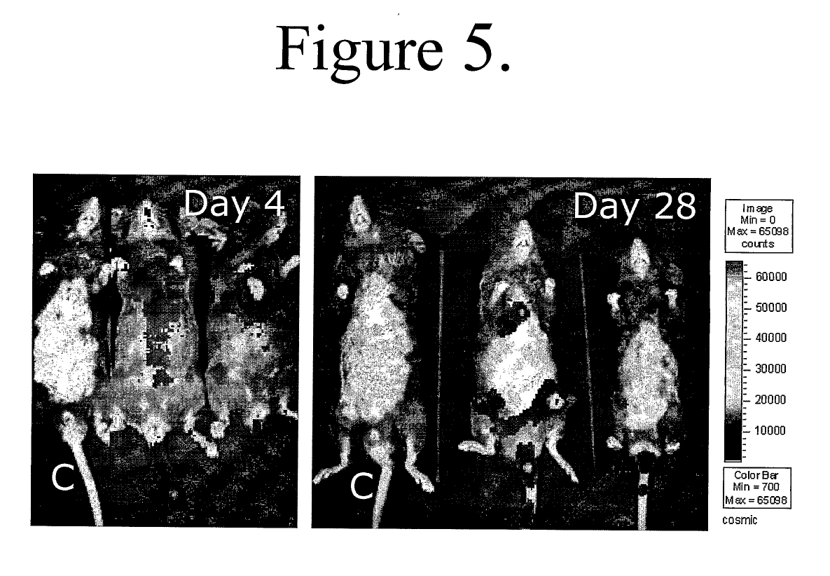

This resistance to MAPCs may be due to an immune response generated to the multiple foreign reporter proteins expressed by MAPC DL (DsRed2, luciferase, neomycin phosphotransferase, β galactosidase) (it is overcome by total body irradiation (TBI) conditioning (FIG. 5), which in turn results in a widespread homing of MAPC).

TABLE 2
MAPC Persistance in Mice with
Various Degrees of Immune Competence

| | | In vivo | Immune | | |
|---|---|---|---|---|---|
| | Genotype | depletion | deficiency | Mean | Range |
| 1 | C57BL/6 | | none | 34 | 27-45 |
| 2 | C57BL/6 | NK1.1 mAb | NK | 40 | 31-63 |
| 3 | Rag2^{-/-} | | T, B | 180 | 37-436 |
| 4 | Rag2^{-/-} | NK1.1 mAb | T, B, NK | 584 | 99-795 |
| 5 | Rag2^{-/-}/γC^{-/-} | | T, B, NK | 762 | 146-3010 |
| 6 | control | N/A | none | 43 | 28-58 |

Luciferase signal was quantified in recipients of MAPC DL 30 days after infusion using BLI technique. Persistence of MAPC DL in six cohorts of mice with various levels of immune competence resulting from either genetic or epigenetic deficiencies is expressed as a mean and range (in photons/second/cm²) of luciferase activity for each cohort.
Control, mice injected with non-labeled MAPC;
mAb, monoclonal antibody;
N/A, not applicable.

Donor MAPCs Engraft in Lung, Liver, and Spleen

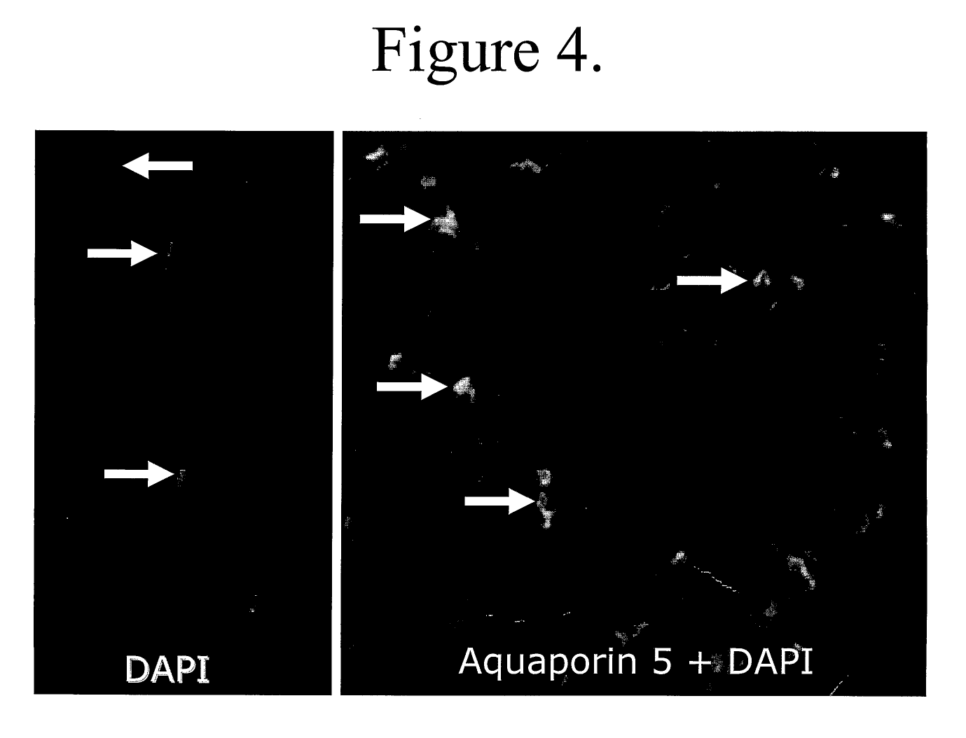

To obtain an assessment of donor MAPC engraftment, lung, liver, and spleen were examined in representative C57BL/6 (N=2 out of 10), Rag2^{-/-} (N=2 out of 10) and Rag2^{-/-}/IL-2Rγc^{-/-} recipients (N=2 out of 6) at day 30 after MAPC DL infusion. Tissue immunohistochemistry revealed MAPC DL cells in all three tissues in all but C57BL/6 wild type mice (data not shown). In lung, MAPC-derived cells not only engrafted in high numbers but also differentiated in alveolar type I pneumocytes (FIG. 4).

Total Body Irradiation Overcomes MAPC Rejection

It was also determined whether MAPCs can persist after TBI conditioning. B10.BR mice were lethally irradiated and given C57BL/6 bone marrow cells with or without 10⁶ of MAPC DL. Bioluminescence signals from donor MAPC-derived cells were detected over the chest, abdomen, head, and extremities of recipient mice from day 4 through day 28 (FIG. 5). This suggests that conditioning for hematopoietic stem cell transplantation using total body irradiation may overcome both NK and T cell mediated resistance, and be advantageous in the long-term survival and widespread homing of MAPCs.

Intra-Areterial Infusion of MAPCs Results in Enhanced Biodistribution

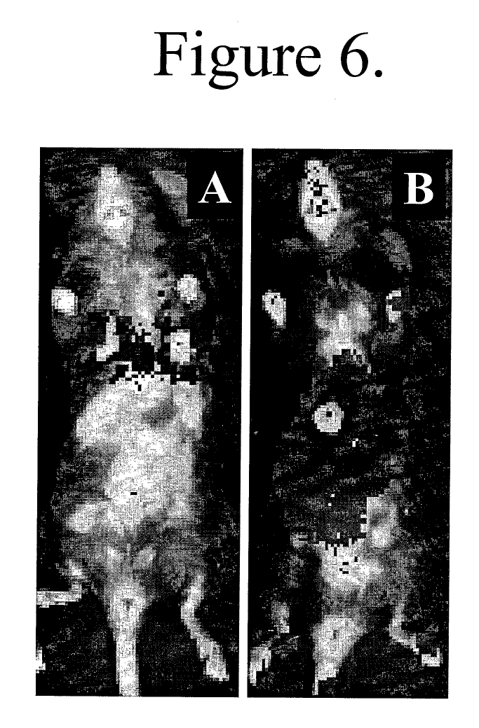

As most of the bioluminescence of infused MAPC DL was detected over the upper thorax, it was reasoned that capture of MAPCs in pulmonary vasculature after intravenous (IV) delivery may decrease the actual MAPC cell dose delivered to other visceral organs. To compare biodistribution of MAPC after intravenous and intra-arterial (IA) delivery, MAPC DL (10⁶) were infused either via tail vein or via left cardiac ventricle into Rag2^{-/-}/IL-2Rγc^{-/-} mice. WBI 10 weeks after either IV or IA infusion showed not only much more diverse homing of MAPCs but also about 10 fold higher total body bioluminescence signals after IA delivery in comparison to the bioluminescence signals observed after IV infusion of the same dose of MAPC DL (data not shown and FIG. 6).

### BIBLIOGRAPHY

Abe, A., et al., J. Virol. 1998; 72: 6159-6163.
Aguila H L, Weissman I L. “Hematopoietic stem cells are not direct cytotoxic targets of natural killer cells.” Blood. 1996; 87:1225-1231.
Akimenko M. A. J Neurosci 1994; 14:3475-86.
Allison, A. C. “Immunosuppressive drugs: the first 50 years and a glance forward.” Immunopharmacology. 2000; 47 (2-3): 63-83.
Auletta J J, Devecchio J L, Ferrara J L, Heinzel F P. “Distinct phases in recovery of reconstituted innate cellular-mediated immunity after murine syngeneic bone marrow transplantation.” Biol Blood Marrow Transplant. 2004; 10:834-847.
Babcook, et al., Proc. Natl. Acad. Sci. (USA). 1996; 93: 7843-7848.
Basch, et. al., J. Immunol. Methods. 1983; 56:269.
Barnes, et al., Purification of Immunoglobulin G (IgG), in: Methods in Molecular Biology, Vol. 10, pages 79-104 (Humana Press (1992).
Batinic, D., et al., Bone Marrow Transplant. 1990; 6(2):103-7.
Benichou, G. “Direct and indirect antigen recognition: the pathways to allograft immune rejection.” Front Biosci. 1999; 4: D476-80.
Ben-Shushan E., et al. Mol Cell Biol 1998; 18:1866-78.
Bevis B J, Glick B S. “Rapidly maturing variants of the Discosoma red fluorescent protein (DsRed).” Nat. Biotechnol. 2002; 20:83-87.
Bird, et al., Science. 1988; 242:423-426.
Bittira B, Shum-Tim D, Al-Khaldi A, Chiu R C. “Mobilization and homing of bone marrow stromal cells in myocardial infarction.” Eur J Cardiothorac Surg. 2003; 24:393-398.
Borue X, Lee S, Grove J, et al. “Bone marrow-derived cells contribute to epithelial engraftment during wound healing.” Am J Pathol. 2004; 165:1767-1772.
Bredenbeek, P. J., et al. J. Virol. 1993; 67:6439-6446.
Buckley, R. “Assessment and modulation of the immune response: transplantation immunology.” Journal of Allergy and Clinical Immunology 2003; 111:1-18.
Cai Z. H., et al., Artif Organs. 1988; 12(5):388-93.
Chang, P., et al., Trends in Biotech. 1999; 17:78-83.
Chang, T. M., Artif Organs. 1992; 16(1):71-4.
Clackson et al. Nature. 1991; 352:624-628.
Clarke, Science. 2000; 288:1660-3.
Clothia et al., J. Mol. Biol. 11985; 186:651-66, 1985.
Coligan, et al., Current Protocols in Immunology, (1991 and 1992).
Davidson, B. L., et al., Nature Genetics. 1993; 3:219-223.
Douglas, J., et al. Nature Biotech. 1999; 17:470-475.
Drukker M, Katz G, Urbach A, et al. “Characterization of the expression of MHC proteins in human embryonic stem cells.” Proc Natl Acad Sci USA. 2002; 99:9864-9869.
Dull, T., et al., J. Virol. 1998; 72:8463-8471.
Edinger M, Hoffmann P, Contag C H, Negrin R S. “Evaluation of effector cell fate and function by in vivo bioluminescence imaging.” Methods. 2003; 31:172-179.
Fandrich, F., et al. “Preimplantation stage stem cells induce long term allogeneic graft acceptance without supplementary host conditioning. Nature Medicine 2002; 8:171-178.
Ferrari, Science. 1998; 279:528-30.
Foerst-Potts L. Dev Dyn 1997; 209:70-84.
Friedenstein A J, Chailakhyan R K, Gerasimov U V. “Bone marrow osteogenic stem cells: in vitro cultivation and transplantation in diffusion chambers.” Cell Tissue Kinet. 1987; 20:263-272.
Friedenstein A J, Gorskaja J F, Kulagina N N. “Fibroblast precursors in normal and irradiated mouse hematopoietic organs.” Exp Hematol. 1976; 4:267-274.
Frolov, I., et al. (Proc. Natl. Acad. Sci. USA. 1996; 93:11371-11377.
Green, et al., Production of Polyclonal Antisera, in: Immunochemical Protocols (Manson, ed.), pages 1-5 (Humana Press).
Gussoni, Nature. 1999; 401:390-4.
Harlow, et al., in: Antibodies: A Laboratory Manual, (Cold Spring Harbor Pub. (1988).
Hofmann, C., et al. J. Virol. 1999; 73:6930-6936.
Hollinger et al., Proc. Natl. Acad. Sci. USA. 1993; 906444-6448 (1993).
Holmes, et al., J. Immunol. 1997; 158:2192-2201.
Ishikawa, H., et al., “CD4⁺ V_{α}14 NKT cells play a crucial role in an early stage of protective immunity against infection with Leishmania major.” International Immunology. 2000; 12(9):1267-1274.
Ivics Z, Hackett P B, Plasterk H, Izsvak Z. “Molecular reconstruction of Sleeping Beauty, a Tc1-like transposon from fish, and its transposition in human cells.” Cell. 1997; 91:501-510.
Jackson, PNAS USA. 1999; 96:14482-6.
Jahagirdar, B. N., et al. Exp Hematol. 2001; 29(5):543-56.
Jiang Y, Henderson D, Blackstad M, Chen A, Miller R F, Verfaillie C M. “Neuroectodermal differentiation from mouse multipotent adult progenitor cells.” Proc Natl Acad Sci USA. 2003; 100 Suppl 1:11854-11860.
Jiang Y, Jahagirdar B N, Reinhardt R L, et al. “Pluripotency of mesenchymal stem cells derived from adult marrow.” Nature. 2002a; 418:41-49.
Jiang Y, Vaessen B, Lenvik T, Blackstad M, Reyes M, Verfaillie C M. “Multipotent progenitor cells can be isolated from postnatal murine bone marrow, muscle, and brain.” Exp Hematol. 2002b; 30:896-904.
Johnston, S. A., et al., Genet. Eng. (NY) 1993; 15: 225-236.
Jones et al., Nature. 1986; 321:522-525.
Kannagi, R. EMBO J. 1983; 2:2355-61.
Katz, S. M. et al., “New immunosuppressive agents.” Transplant Proc. 2000; 32(3): 620-1.
Kawada H, Fujita J, Kinjo K, et al. “Nonhematopoietic mesenchymal stem cells can be mobilized and differentiate into cardiomyocytes after myocardial infarction.” Blood. 2004; 104:3581-3587.
Kim S, Iizuka K, Kang H S, et al. “In vivo developmental stages in murine natural killer cell maturation.” Nat Immunol. 2002; 3:523-528.
Kohler & Milstein, Nature. 1975; 256:495.
Krause D S, Theise N D, Collector M I, et al. “Multi-organ, multi-lineage engraftment by a single bone marrow-derived stem cell.” Cell. 2001; 105:369-377.
Lagasse E, Connors H, Al-Dhalimy M, et al. “Purified hematopoietic stem cells can differentiate into hepatocytes in vivo.” Nat. Med. 2000; 6:1229-1234.
Lanier L L. “NK Cell Recognition.” Annu Rev Immunol. 2004.
Laquerre, S., et al. J. Virol. 1998; 72:9683-9697.
Larrick, et al., Methods: A Companion to Methods in Enzymology. (1991).
Lawrence H. Blood 1997; 89:1922.
Le Blanc K, Rasmusson I, Gotherstrom C, et al. “Mesenchymal stem cells inhibit the expression of CD25 (interleukin-2 receptor) and CD38 on phytohaemagglutinin-activated lymphocytes.” Scand J Immunol. 2004a; 60:307-315.
Le Blanc K, Rasmusson I, Sundberg B, et al. “Treatment of severe acute graft-versus-host disease with third party haploidentical mesenchymal stem cells.” Lancet. 2004b; 363:1439-1441.
Lefebvre V. Matrix Biol 1988; 16:529-40.
Lim, J. W. and Bodnar, A., Proteomics. 2002; 2(9):1187-1203 (2002).
Loeffler, J. and Behr, J., Methods in Enzymology. 1993; 217:599-618.
Kafri, T., et al., J. Virol. 1999; 73:576-584.
Mackenzie T C, Flake A W. “Human mesenchymal stem cells persist, demonstrate site-specific multipotential differentiation, and are present in sites of wound healing and tissue regeneration after transplantation into fetal sheep.” Blood Cells Mol Dis. 2001; 27:601-604.
Majumdar M K, Keane-Moore M, Buyaner D, et al. “Characterization and functionality of cell surface molecules on human mesenchymal stem cells.” J Biomed Sci. 2003; 10:228-241.
Mammolenti M, Gajavelli S, Tsoulfas P, Levy R. “Absence of major histocompatibility complex class I on neural stem cells does not permit natural killer cell killing and prevents recognition by alloreactive cytotoxic T lymphocytes in vitro.” Stem Cells. 2004; 22:1101-1110.
Marks et al., J. Mol. Biol. 1991; 222:581-597.
Martin, F., et al., J. Virol. 1999; 73:6923-6929.
Matthew, H. W., et al., ASAIO Trans. 1991; 37 (3):M328-30.
Miller, A. D., and C. Buttimore, Mol. Cell. Biol. 1986; 6:2895-2902.
Mochizuki, H., et al., J. Virol. 1998; 72:8873-8883.
Molin, M., et al. J. Virol. 1998; 72:8358-8361.
Morrison et al. Proc. Natl. Acad. Sci. 1984; 81, 6851-6855.
Murphy W J, Kumar V, Bennett M. “Acute rejection of murine bone marrow allografts by natural killer cells and T cells. Differences in kinetics and target antigens recognized.” J Exp Med. 1987; 166:1499-1509.
Muschler, G. F., et al. J Bone Joint Surg. Am. 1997; 79(11):1699-709.
Nichols J., et al. Cell. 1998; 95:379-91.
Novotny and Haber, Proc. Natl. Acad. Sci. USA. 1985; 82:4592-4596.
Offield M. F. Development 1996; 122:983-95.
Pack, et al., Bio/Technology. 1993; 11:1271-77.
Packer A. I. Dev Dyn 2000; 17:62-74.
Persons, D., et al., Nature Medicine. 1998; 4: 1201-1205.
Petersen, Science. 1999; 284:1168-1170.
Pittenger M F, Mackay A M, Beck S C, et al. “Multilineage potential of adult human mesenchymal stem cells.” Science. 1999; 284:143-147.
Pluckthun in The Pharmacology of Monoclonal Antibodies, vol. 113, Rosenburg and Moore eds. Springer-Verlag, N.Y. 1994; pp. 269-315.
Potian J A, Aviv H, Ponzio N M, Harrison J S, Rameshwar P. “Veto-like activity of mesenchymal stem cells: functional discrimination between cellular responses to alloantigens and recall antigens.” J Immunol. 2003; 171:3426-3434.
Presta, Curr. Op. Struct. Biol. 1992; 2:593-596.
Prlic M, Blazar B R, Khoruts A, Zell T, Jameson S C. “Homeostatic expansion occurs independently of costimulatory signals.” J Immunol. 2001; 167:5664-5668.
Rasmusson I, Ringden O, Sundberg B, Le Blanc K. “Mesenchymal stem cells inhibit the formation of cytotoxic T lymphocytes, but not activated cytotoxic T lymphocytes or natural killer cells.” Transplantation. 2003; 76:1208-1213.
Raulet D H. “Interplay of natural killer cells and their receptors with the adaptive immune response.” Nat. Immunol. 2004; 5:996-1002.
Reichmann et al., Nature. 1988; 332:323-329.
Reyes M, Dudek A, Jahagirdar B, Koodie L, Marker P H, Verfaillie C M. “Origin of endothelial progenitors in human postnatal bone marrow.” J Clin Invest. 2002; 109:337-346.
Reyes M, Verfaillie C M. “Characterization of multipotent adult progenitor cells, a subpopulation of mesenchymal stem cells.” Ann N Y Acad Sci. 2001; 938:231-233; discussion 233-235.
Robbins, et al. J. Virol. 1997; 71(12):9466-9474.
Rosfjord E. and Rizzino A. Biochem Biophys Res Commun. 1997; 203:1795-802.
Salmons, B. and Gunzburg, W. H., “Targeting of Retroviral Vectors for Gene Therapy,” Hum. Gene Therapy. 1993; 4:129-141.
Sebestyen, et al. Nature Biotech. 1998; 16:80-85.
Schwartz R E, Reyes M, Koodie L, et al. “Multipotent adult progenitor cells from bone marrow differentiate into functional hepatocyte-like cells.” J Clin Invest. 2002; 109:1291-1302.
Schwarzenberger, P., et al., J. Virol. 1997; 71:8563-8571.
Seino K, Taniguchi M. “Functional roles of NKT cell in the immune system.” Front Biosci. 2004; 9:2577-2587.
Shimozaki et al. Development. 2003; 130:2505-12.
Sutton, R., et al., J. Virol. 1998; 72:5781-5788.
Takahashi, Nat. Med. 1999; 5:434-8.
Takahashi, J Clin Invest. 2000; 105:71-7.
Theise, Hepatology. 2000a; 31:235-40.
Theise, Hepatology. 2000b; 32:11-6.
Tse W T, Pendleton J D, Beyer W M, Egalka M C, Guinan E C. “Suppression of allogeneic T-cell proliferation by human marrow stromal cells: implications in transplantation.” Transplantation. 2003; 75:389-397.
Uwanogho D. et al., Mech Dev. 1995; 49:23-26.
Vaswani, et al., Annals Allergy, Asthma & Immunol. 1998; 81:105-115.
Verfaillie, C. M. Trends Cell Biol. 2002; 12(11):502-8.
Wagers A J, Sherwood R I, Christensen J L, Weissman I L. “Little evidence for developmental plasticity of adult hematopoietic stem cells.” Science. 2002; 297:2256-2259.
Wagner, E., et al., Proc. Natl. Acad. Sci. USA. 1992; 89:6099-6103.
Waldman, H, et al. “Exploiting tolerance processes in transplantation.” Science. 2004; 305:209-212.
Williams, R. S., et al., Proc. Natl. Acad. Sci. USA. 1991; 88:2726-2730.
Whitlow, et al., Methods: A Companion to Methods in Enzymology (1991).
Wold, W., Adenovirus Methods and Protocols, Humana Methods in Molecular Medicine (1998), Blackwell Science, Ltd.
Wu G D, Nolta J A, Jin Y S, et al. “Migration of mesenchymal stem cells to heart allografts during chronic rejection.” Transplantation. 2003; 75:679-685.
Wysocki and Sato, Proc. Natl. Acad. Sci. (USA). 1978; 75:2844.
Xiong, C., et al., Science. 1989; 243:1188-1191.
Yanagi, K., et al., ASAIO Trans. 1989; 35(3):570-2.
Yang, N. S., et al., Proc. Natl. Acad. Sci. USA. 1990; 87:9568-9572.
Zhao L R, Duan W M, Reyes M, Keene C D, Verfaillie C M, Low W C. “Human bone marrow stem cells exhibit neural phenotypes and ameliorate neurological deficits after grafting into the ischemic brain of rats.” Exp Neurol. 2002; 174:11-20.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to increase persistence of MHC-I negative cells comprising administering a population of the MHC-I negative cells and an effective amount of a means for inhibiting Natural Killer cell function to a subject, so that persistence of the MHC-I negative cells increases compared to the method without administration of the inhibiting means.

2. A method to increase engraftment of MHC-I negative cells comprising administering a population of the MHC-I negative cells and an effective amount of a means for inhibiting Natural Killer cell function to a subject, so that engraftment of the MHC-I negative cells increases compared to the method without administration of the inhibiting means.

3. A method to increase immunologic tolerance in a subject to MHC-I negative cells comprising administering a population of the MHC-I negative cells and an effective amount of a means for inhibiting Natural Killer cell function to the subject, so that immunologic tolerance to the MHC-I negative cells increases compared to the method without administration of the inhibiting means.

4. A method to inhibit rejection of MHC-I negative cells comprising administering a population of the MHC-I negative cells and an effective amount of a means for inhibiting Natural Killer cell function to a subject in need thereof, so that rejection of the MHC-I negative cells is inhibited in comparison to the method without administration of the inhibiting means.

5. A method for treating a disease or injury in a subject comprising administering to a subject an effective amount of a population of MHC-I negative cells and an effective amount of a means for inhibiting Natural Killer cell function.

6. The method of claim 3, wherein the MHC-I negative cells are non-ES, non-EG, non-germ cells, wherein the non-ES, non-EG, non-germ cells and can differentiate into ectodermal, endodermal and mesodermal cell types.

7. The method of claim 6, wherein the non-ES, non-EG, non-germ cells are autologous, allogeneic or xenogeneic to the subject.

8. The method claim 3, wherein the MHC-I negative cells are embryonic stem cells.

9. The method of claim 3, wherein the MHC-I negative cells are administered by localized injection, catheter administration, systemic injection, intraperitoneal injection, parenteral administration, oral administration, intracranial injection, intra-arterial injection, intravenous injection, intraventricular infusion, intraplacental injection, intrauterine injection, surgical intramyocardial injection, transendocardial injection, intracoronary injection, transvascular injection, intramuscular injection or via direct application to tissue surfaces during surgery or on a wound.

10. The method claim 3, wherein the means to inhibit Natural Killer cell function is an anti-Natural Killer cell antibody or an active fragment thereof.

11. The method of claim 10, wherein the antibody is a polyclonal antibody.

12. The method of claim 10, wherein the antibody is a monoclonal antibody.

13. The method of claim 3 wherein the means to inhibit Natural Killer cell function is a pharmaceutical.

14. The method of claim 3 wherein the means to inhibit Natural Killer cell function is a protein.

15. The method of claim 3, wherein the means to inhibit Natural Killer cell function is total body irradiation.

16. The method of claim 15, wherein the total body irradiation is administered as a non-lethal dose.

17. The method of claim 3, wherein the means to inhibit Natural Killer cell function is administered prior to, during, after or a combination thereof, administration of the MHC-I negative cells.

18. The method of claim 3, wherein the means to inhibit Natural Killer cell function is administered locally at the site of engraftment, systemically or a combination thereof.

19. The method of claim 3 further comprising administration of bone marrow.

20. The method of claim 3 further comprising a secondary transplant.

21. The method of claim 20, wherein the secondary transplant is a heart, lung, kidney, liver, bone marrow transplant or a combination thereof.

22. The method of claim 3, wherein the subject is suffering from a disease or injury.

23. The method of claim or 22, wherein the disease is a cardiac disorder, cancer, autoimmune disease, genetic disease or hematological disease.

24. The method claim or 22, wherein the injury is a result of total body irradiation, chemoradiotherapy or physical trauma.

25. A composition comprising a means for inhibiting NK cell function, a population of MHC-I negative cells and a pharmaceutically acceptable carrier.

26-28. (canceled)
